SEQUENCE LISTING (16 sequences)

SEQ ID NO: 1 - protein, 619 aa, Streptococcus pneumoniae
MNKKKMILTSLASVAILGAGFVASQPTVVRAEESPVASQSKAEKDYDAAKKDAKNAKKAVEDAQKALDDAKAAQKKYDEDQKKTEEKAALEKAASEEMDKAVAAVQQAYLAYQQATDKAAKDAADKMIDEAKKREEEAKTKFNTVRAMVVPEPEQLAETKKKSEEAKQKAPELTKKLEEAKAKLEEAEKKATEAKQKVDAEEVAPQAKIAELENQVHRLEQELKEIDESESEDYAKEGFRAPLQSKLDAKKAKLSKLEELSDKIDELDAEIAKLEDQLKAAEENNNVEDYFKEGLEKTIAAKKAELEKTEADLKKAVNEPEKPAPAPETPAPEAPAEQPKPAPAPQPAPAPKPEKPAEQPKPEKTDDQQAEEDYARRSEEEYNRLTQQQPPKAEKPAPAPKTGWKQENGMWYFYNTDGSMATGWLQNNGSWYYLNSNGAMATGWLQYNGSWYYLNANGAMATGWAKVNGSWYYLNANGAMATGWLQYNGSWYYLNANGAMATGWAKVNGSWYYLNANGAMATGWLQYNGSWYYLNANGAMATGWAKVNGSWYYLNANGAMATGWVKDGDTWYYLEASGAMKASQWFKVSDKWYYVNGLGALAVNTTVDGYKVNANGEWV

SEQ ID NO: 2 - protein, 40 aa, Artificial Sequence
  stabilizing strand
CXGGGEIEALKKEIEALKKEIEALKKEIEALKKEIEALKK

SEQ ID NO: 3 - protein, 38 aa, Artificial Sequence
  hybrid sequence
CXGIEELEKKITELKQKIDALENQIHRLEQEIKELDES

SEQ ID NO: 4 - protein, 38 aa, Artificial Sequence
  hybrid sequence
CXGLEEAEKKATEAKQKVDALENQVHRLEQELKEIDES

SEQ ID NO: 5 - protein, 20 aa, Artificial Sequence
  consensus sequence
EELXXKIDELDXEIAXLEKX

SEQ ID NO: 6 - protein, 8 aa, Artificial Sequence
  consensus sequence
EELSDKID

SEQ ID NO: 7 - protein, 27 aa, Artificial Sequence
  hybrid sequence
CXGEIEALKKKIEELSDKIDELEKEIK

SEQ ID NO: 8 - protein, 21 aa, Haemophilus influenzae
IKKVLEIGLNMSQEASNLTSA

SEQ ID NO: 9 - protein, 27 aa, Artificial Sequence
  hybrid sequence
EAEIKKLLEIILNLSQEISNLTSALKG

SEQ ID NO: 10 - protein, 20 aa, Streptococcus pneumoniae
LEKDVEDFKNSDGEQAEQVL

SEQ ID NO: 11 - protein, 15 aa, Streptococcus pneumoniae
LEDNLKDAETNNVED

SEQ ID NO: 12 - protein, 10 aa, Streptococcus pneumoniae
LAKKQTELEK

SEQ ID NO: 13 - protein, 35 aa, Artificial Sequence
  hybrid sequence
DVEDFKNSDGELEDNLKDAETNNVEDAKKQTELEK

SEQ ID NO: 14 - protein, 35 aa, Artificial Sequence
  hybrid sequence
DIEDLKNSIGELEDNIKELETNIVELAKKITELEK

SEQ ID NO: 15 - protein, 7 aa, Artificial Sequence
  Coiled-coil scaffold
XXXXXXX

SEQ ID NO: 16 - protein, 10 aa, Artificial Sequence
  Coiled-coil scaffold
CXGIXXLXXX

(19) United States
(12) Patent Application Publication
(10) Pub. No.: US 2003/0021795 A1
(43) Pub. Date: Jan. 30, 2003

(54) Use of coiled-coil structural scaffold to generate structure-specific peptides

(76) Inventors: Michael E. Houston, Edmonton (CA); Robert Hodges, Denver, CO (US)

Correspondence Address: BURNS DOANE SWECKER & MATHIS L L P, POST OFFICE BOX 1404, ALEXANDRIA, VA, 22313-1404, US

(21) Appl. No.: 09/882,774

(22) Filed: Jun. 14, 2001

Related U.S. Application Data
(60) Provisional application No. 60/211,892, filed on Jun. 14, 2000.
(60) Provisional application No. 60/213,387, filed on Jun. 23, 2000.

(51) Int. Cl. A61K 39/00; C07K 7/06; C07K 7/08; C07K 14/00

(52) U.S. Cl. 424/185.1; 530/324; 530/325; 530/326; 530/327; 530/328

(57) ABSTRACT

This invention relates to the use of a coiled-coil structural scaffold to generate structure-specific peptides, including synthetic peptides derived from naturally occurring proteins of various origin. The structure of the synthetic peptides utilizes a scaffold of heptad repeat units into which epitopes from coiled-coil regions of native proteins are spliced. In particular, the synthetic peptides may be based on microbial proteins, especially surface proteins, which occur naturally in the coiled-coil form such as pneumococcal surface proteins A and C. The synthetic peptides are immunogenic and can be used to elicit an immune response in an animal. Accordingly, they are useful as vaccines or to stimulate antibody production or cell-mediated immunity to the naturally occurring protein.

### RELATED APPLICATIONS

This application claims the benefit of U.S. Provisional Applications Ser. No. 60/211,892, filed Jun. 14, 2000, and Ser. No. 60/213,387, filed Jun. 23, 2000. The entire content of each of these provisional applications is hereby incorporated by reference in its entirety.

### FIELD OF THE INVENTION

This invention relates to the use of a coiled-coil structural scaffold to generate structure-specific peptides, including synthetic peptides derived from naturally occurring microbial and non-microbial protein antigens. The structure of the synthetic peptides utilizes a scaffold of heptad repeat units into which epitopes derived from coiled-coil regions of native proteins are spliced. The resulting peptide has a more stable coiled-coil structure, hence improving presentation of the epitopes in a helical conformation.

References

U.S. Pat. No. 6,075,181.

U.S. Pat. No. 6,150,584.

WO 96/11944, published Apr. 25, 1996.

WO 01/00010, published Jan. 4, 2001.

Alexander J. E., et al. Infect. Immun. 62: 5693-5688 (1994).

Baquero, F., et al. J. Antimicrob. Chemother. 28 (Suppl. C): 31-38 (1991).

Beuvery, E. C., et al. Infect. Immun. 37: 15-22 (1982).

Briles D. E. et al. Infect. Immun. 57(5): 1457-1464 (1989).

Briles, D. E. et al. Pneumococcal diversity: considerations for new vaccine strategies with emphasis on pneumococcal surface protein A (PspA). Clin. Microbiol. Rev. 11(4):645-657 (1998).

Briles, D. E., et al. In Effects of microbes on the immune system (Cunningham, M. W., Flinami, R. S. Eds) Philadelphia: LippinconRaven: pp 263-280 (1999).

Briles, D. E., et al. Vaccine 18: 1707-1711 (2000).

Brooks-Walter, A., et al. Infect. Immun. 67: 6533-6542 (1999).

Canvin, J. R., et al. J. Infect. Dis. 172: 119-123 (1995).

Chang K, Pastan I. “Molecular cloning and expression of a cDNA encoding a protein detected by the K1 antibody from an ovarian carcinoma (OVCAR-3) cell line”, Int J. Cancer. 57(1):90-7 (1994).

Cowan, M. J., et al. Pediatrics 62: 721-727 (1978).

Crain M. J., et al. Microb. Pathog. 21: 265-275 (1996).

Davis, S., Aldrich, T. H., Jones, P. F., Acheson, A., Compton, D. L., Jain, V., Ryan, T. E., Bruno, J., Radziejewski, C., Maisonpierre, P. C. and Yancopoulos, G. D. Isolation of angiopoietin-1, a ligand for the TIE2 receptor, by secretion-trap expression cloning. Cell 87 (7): 1161-1169 (1996).

De, B. K., et al. Pathobiology 67: 115-122 (1999).

Henrichsen, J., J. Clin. Microbiol. 33: 2759-2762 (1995).

Houston, M. E. et al. Biochemistry 35: 10041-10050 (1996).

Kalin, M. Pneumococcal serotypes and their clinical relevance. Thorax 53: 159-162 (1998).

Kapanadze B, Kashuba V, Baranova A, Rasool O, van Everdink W, Liu Y, Syomov A, Corcoran M, Poltaraus A, Brodyansky V, Syomova N, Kazakov A, Ibbotson R, van den Berg A, Gizatullin R, Fedorova L, Sulimova G, Zelenin A, Deaven L, Lehrach H, Grander D, Buys C, Oscier D, Zabarovsky E R, Yankovsky N. A cosmid and cDNA fine physical map of a human chromosome 13q14 region frequently lost in B-cell chronic lymphocytic leukemia and identification of a new putative tumor suppressor gene, Leu5. FEBS Lett. 426(2):266-70 (1998).

Lancet, Lancet 2: 699-701 (1985).

Lawrence, M. C., et al. Structure 6: 1553-1661 (1998).

Li Y, Kang J, Friedman J, Tarassishin L, Ye J, Kovalenko A, Wallach D, Horwitz M S. Identification of a cell protein (FIP-3) as a modulator of NF-kappaB activity and as a target of an adenovirus inhibitor of tumor necrosis factor alpha-induced apoptosis. Proc Natl Acad Sci USA. 96(3):1042-7 (1999).

Li, L., Li, X., Francke, U. and Cohen, S. N. The TSG101 tumor susceptibility gene is located in chromosome 11 band p15 and is mutated in human breast cancer. Cell 88 (1): 143-154 (1997).

McDaniel. L. S., et al. Microb. Pathog. 17: 323-337 (1994).

McDaniel. L. S., et al. Infect. Immun. 59: 222-228 (1991).

Michon F., et al. Vaccine 16: 1732-41 (1998).

Miranda C, Minoletti F, Greco A, Sozzi G, Pierotti M. A. “Refined localization of the human TPR gene to chromosome 1q25 by in situ hybridization”, Genomics 23(3):714-5 (1994).

Nabors, G. S., et al. Vaccine 18: 1743-1754 (2000).

Nakashima, M., Sonoda, K. and Watanabe, T. Inhibition of cell growth and induction of apoptotic cell death by the human tumor-associated antigen RCAS1. Nat. Med. 5 (8): 938-942 (1999).

Remington's Pharmaceutical Sciences, Mack Publishing Company, Philadelphia Pa. 18^{th} ed. (1990).

Robbins, J. B., et al. J. Infect. Dis. 148: 1136-1159 (1983).

Shapiro, E. D., et al. N. Engl. J. Med. 325: 1453-1460 (1991).

Shelly, M. A., et al. Infect. Immun. 65: 242-247 (1997).

Singh et al., “Advances in vaccine adjuvants”, Nat. Biotechnol. 17(11):1075-81 (1999).

Spika, J. S., et al. J. Infect. Dis. 163: 1273-1278 (1991).

Takala. A. K., et al. J. Infect. Dis. 164: 982-986 (1991).

Talkington, D. F., et al. Infect. Immun. 59: 1285-1289 (1991).

Talkington, D. F., et al. Microb, Pathog. 21: 17-22 (1996).

Tart, R. C., et al. J. Infect. Immun. 173: 380-386 (1996).

Tripet, B. et al., J. Mol. Biol. 300: 377-402 (2000).

Wagschal, K. et al., Protein Science 8: 2312-2329 (1999).

Winter et al., Nature 349: 293 (1991).

Wolf, E. et al. MultiCoil: A program for predicting two- and three-stranded “coiled coils”. Protein Science 6: 1179-1189 (1997).

World Health Organization. WHO 9 (1995).

Yamada et al., “Identification of a novel autoantigen UACA in patients with panuveitis”, Biochem. Biophys. Res. Commun. 280(4):1169-76 (2001).

All of the above publications, patents and patent applications are herein incorporated by reference in their entirety to the same extent as if the disclosure of each individual publication, patent application or patent was specifically and individually indicated to be incorporated by reference in its entirety.

### BACKGROUND OF THE INVENTION

Streptococcus pneumoniae is an important pathogen causing life-threatening invasive diseases such as pneumonia, meningitis and bacteraemia. It is estimated that more than 1 million cases of pneumococcal pneumonia are diagnosed each year in the United States with 0.7% of these infections being fatal (Lancet, 1985). In developing countries, it is estimated that 4 million deaths are due to pneumonia, with S. pneumoniae infections accounting for 70% of the deaths (WHO, 1995). S. pneumoniae also causes less serious diseases such as otitis media and sinusitis which, due to their prevalence, are a significant burden on health care systems. The high morbidity and mortality rate associated with pneumococcal infections is exacerbated by the rate at which the organism acquires resistance to multiple antibiotics (Spika et al., 1991; Baquero et al., 1991). Thus, there is an unmet need for effective treatments for pneumococcal infections.

The current vaccine utilized for prevention of pneumococcal infection in humans is based on purified capsular polysaccharides. The design of a capsular polysaccharide vaccine is complicated by the fact that there are 90 different capsular types and the protection elicited by the capsule is type specific (Henrichson, 1995). These problems are somewhat mitigated by the fact that certain capsules are more commonly associated with human disease than others and which ultimately led to their inclusion into the current 23 valent vaccine (Robbins et al., 1983). However, capsular polysaccharides, like most polymeric compounds possessing multiple repeating units, are inefficient in stimulating immune responses and subclass switching to IgG (Beuvery et al., 1982). Not surprisingly, the vaccine is only 60% effective in preventing fatal pneumococcal bacteraemia in the elderly (Shapiro et al., 1991) and is unable to elicit adequate antibody responses in children under the age of 2 (Cowan et al., 1978). The problem of poor immunogenicity in children is being addressed by conjugation of the polysaccharides to protein carriers such as diphtheria and tetanus toxoids (Shelly et al., 1997). In a recent clinical trial, a seven valent conjugate vaccine was shown to be immunogenic in children and elicited protection against invasive pneumococcal infection.

However, a separate vaccine will be required for adults because clinically important infections are caused by different capsular types than is the case for children. Coverage in the developing world is expected to be as low as 52% owing to geographical variation of pneumococcal strains. In addition, it is anticipated that the conjugate vaccines may be too expensive for the developing world. Thus, there is an urgent need for alternative approaches to the development of pneumococcal vaccines.

The drawbacks associated with the polysaccharide vaccine have prompted interest in the possibility of developing vaccines based on pneumococcal protein antigens. Pneumococcal proteins under consideration have included the toxins, pneumolysin, autolysin, and the surface proteins. The surface proteins which have been used as vaccines to date include pneumococcal surface adhesion A (PsaA), pneumococcal surface protein A (PspA) and pneumococcal surface protein C (PspC). The surface proteins are shown in FIG. 1.

Pneumolysin is a thiol cytolysin that is produced by virtually all clinical isolates of S. pneumoniae. When mice were immunized with a genetically engineered toxoid version of pneumolysin (pneumolysoid) absorbed onto alum, the survival rate and the time of death of non-survivors (survival time) was significantly greater than that of controls. However, significant differences in the degree of protection were noted between different strains (Alexander et al., 1994). Pneumolysoid is currently being investigated as a carrier protein for pneumococcal capsular polysaccharides (Michon et al., 1998). In general, a higher response to the capsular polysaccharides was observed when they are formulated as conjugates of pneumolysin as compared to tetanus toxoid conjugates. Immunization with autolysin has also shown to be protective in a sepsis model (Canvin et al., 1995). However, antibodies to autolysin appear to exert their effect in part because they prevent the autolysin dependent release of pneumolysin.

All Streptococcus pneumoniae isolates investigated to date express the lipoprotein pneumococcal surface adhesin A (PsaA). PsaA is 37 kDa in molecular weight and is thought to be genetically conserved. The function of PsaA is that of a Zn binding membrane transport protein (Lawrence, et al. 1998). Recent studies have shown that immunization of mice with native PsaA elicited anti-PsaA antibodies that protected mice from challenge with virulent strains of S. pneumoniae (Talkington et al., 1996; De et al., 1999). In a mouse carriage model, PsaA has been shown to be one of the best pneumococcal surface antigens at eliciting protection against carriage. However, PsaA is not as effective as PspA in eliciting protection in a murine sepsis model or murine pulmonary infection model (Briles et al., 2000).

Pneumococcal surface protein A has been found on all strains of S. pneumoniae studied to date and is required for full virulence (Crain et al., 1990). While PspA has a strain-dependent molecular weight ranging from 67 to 99 kDa, all proteins consist of four distinct domains: an N-terminal highly charged domain, a proline rich domain, a repeat domain comprising 10 highly conserved 20 amino acid repeats, and a short hydrophobic domain (FIG. 2). The repeat region is responsible for attachment of the protein to the cell surface of S. pneumoniae by non-covalent binding to choline residues.

The amino acid sequence of the R×1 strain PspA is included herein as SEQ ID NO: 1. Analysis of the sequence of the N-terminus of this PspA (residues 1-303) indicated that the sequence adopts a coiled-coil structure. The coiled-coil structure consists of two amphipathic α-helices wrapped around each other with a left handed supertwist (FIG. 3). The coiled-coil is characterized by a heptad repeat (abcdefg), in which the a and d positions are typically occupied by hydrophobic amino acids. These residues are aligned such that they create a hydrophobic face that is responsible for the stability of this structure.

The N-terminal sequence of PspA contains three prolines interspersed throughout the sequence, which are believed to create small breaks in the otherwise continuous coiled-coil. It is proposed from hydrodynamic characterization of the protein and from computer modeling that PspA forms a coiled-coil by folding back onto itself at one of the three proline regions. It is thought that the coiled-coil region extends from the cell wall and possibly protrudes outside the capsule.

The exact function of PspA is not known. It has been shown to retard to clearance of pneumococci from the blood (McDaniel et al., 1987). It seems likely that the major effect of PspA on virulence may be due to interference with complement fixation. In addition, it is thought that the highly positively charged nature of the coiled-coil region may interact with the negatively charged capsule. This interaction may stabilize the capsular structure.

Streptococcus pneumoniae has another surface protein called PspC that has a similar domain structure as PspA, but has a larger coiled-coil region and is larger in overall size (FIG. 2). The N-terminal 150 amino acids of the helical domain are highly variable in size and sequence. Adjacent to this hypervariable region are two repeating sequences that vary in size from 101 to 205 amino acids in length. In the larger PspC proteins, there exists a helical region similar to the C-terminal portion of the coiled-coil region found in PspA. A comparison of PspC and PspA proteins is shown in FIG. 2. Evidence now exists that most strains of S. pneumoniae produce both PspA and PspC (Brooks-Walter et al., 1999; Crain et al., 1996). The exact function of PspC is not known, but it is believed to function in an analogous fashion as PspA.

### SUMMARY OF THE INVENTION

This invention relates to the unexpected discovery that the characteristic heptad repeat (abcdefg)ₙ of coiled-coil proteins, where the a and d positions are typically occupied by hydrophobic amino acids, can be used as a template to lock epitopes from coiled-coil regions or potential coiled-coil regions of native proteins into a stable coiled-coil structure to form novel synthetic peptides based on native proteins. Such peptides may be used for immunization. Amino acid residues from corresponding positions of native coiled-coil proteins are spliced into the solvent exposed b, c, e, f and g positions, while hydrophobic amino acids (in particular, Ile and Leu) occupy the a and d positions. The resulting novel peptides maintain a stable coiled-coil structure in aqueous solution, and present immunogenic epitopes characteristic of the native proteins.

Accordingly, one aspect of the invention provides for synthetic peptides of the following formula (formula I):

| | | |
|---|---|---|
| (AXXDXXX)ₙ | I | (SEQ ID NO:15) |

wherein A is Ile, Leu, Val or a derivative thereof, D is Leu, Ile, Val or a derivative thereof, each X is an amino acid residue or derivative thereof which corresponds to an amino acid residue of an epitope of a native coiled-coil protein to form a set of X residues, and n is an integer equal to or greater than 1. Preferably, A is Ile and D is Leu in every set of the (AXXDXXX) repeat. Preferred values for n are 2 to 100. In a preferred embodiment, n is 2 to 10, most preferably about 3 to 6.

Preferred sets of X residues are amino acids that are solvent exposed in the coiled-coil region of the native protein. Each set of X amino acid residues may be selected and/or combined independently. In other words, they may come from the same epitope of the same protein, different epitopes of the same protein, epitopes of different proteins, or the like.

The peptides may further comprise additional amino acids, such as the amino acid residues CNleG-, at the N-terminus of the peptide. The peptides may also comprise additional amino acids at the C-terminus. The additional amino acids may or may not be arranged in a sequence which has the propensity to form coiled-coils, and the number of additional amino acids at either terminus is preferably less than 100. The number of additional amino acids at either terminus is more preferably less than 50, yet more preferably less than 30, still more preferably less than 20, and most preferably less than 10.

This invention may particularly be used to create novel synthetic peptides relating to microbial proteins which exist as coiled-coil structures in the native state. In particular, amino acid residues from epitopes of microbial surface proteins, most particularly S. pneumoniae surface proteins A and C, may be used in the novel peptides. Many microbial surface proteins have similar architecture, existing as long, free-standing coiled-coils.

The X residues may come from consensus sequences of native coiled-coil proteins, for example PspA and PspC. A preferred consensus sequence of PspA is EELX₁X₂KIDELDX₃EIAX₄LEKX₅ (SEQ ID NO: 5). Preferably, X₁ is S, Q, N or D; X₂ is D, N or K; X₃ is A or N; X₄ is K, E or D; and X₅ is N, D or E.

To increase the stability of the coiled-coil conformation, the amino acid residues at the e and g positions, if not part of the epitope of interest, may optionally be replaced with charged amino acids (e.g., Asp, Glu, Lys, Arg and His). The replacement should be performed such that the e position in one peptide strand and the g′ position in the “complementary strand” are occupied by amino acids of opposite charges, for example Asp and Lys¹. Similarly, the residues at the g position of one strand and the e′ position of the complementary strand can be replaced by residues of opposite charges. ≠¹For example, chain 1 and chain 2 in FIG. 3 are “complementary strands”. As illustrated in FIG. 3, the e position in chain 1 and the g′ position in chain 2 are aligned. Therefore, if amino acid residues with opposite charge occupy these two positions, they can form a salt bridge between their side chains, which increases the stability of the coiled-coil structure.

The residues may also be replaced by charged amino acids such that a salt bridge can form between two residues which are 3 or 4 residues apart in the same strand. Alternatively, a charged amino acid may stabilize the coiled-coil structure by interacting with an oppositely-charged residue in a stabilizing strand.

In another aspect, the invention provides methods of making peptides of the formula I comprising the steps of selecting an antigenic epitope derived from a coiled-coil, determining which amino acid residues are solvent exposed, and inserting said solvent exposed amino acid residues into the X positions of formula I. In particular, the selection of epitopes may be performed using a computer algorithm. More than one set of epitopic amino acids derived from the same protein or different proteins may be used. Furthermore, the sets of epitopic amino acids may be from different strains and/or species of microorganism when epitopes of a microbial coiled-coil protein is used.

In a further aspect, the invention provides compositions useful to stimulate an immune response in an animal wherein said compositions comprise a peptide of formula I. When more than one set of epitopic amino acids is used in the peptide of formula I and the sets of epitopic amino acids are from different strains and/or species of microorganism, the composition is useful to stimulate an immune response to more than one strain and/or species of microorganism. The animal may be an avian, a mammal (including a human), or any animal capable of an immune response.

Antibodies raised by administration of a peptide of formula I are also provided as an aspect of the invention. Such antibodies may be polyclonal or monoclonal. Pharmaceutical compositions comprising such antibodies are also provided. When more than one set of epitopic amino acids is used in the peptide of formula I and the sets of epitopic amino acids are from different strains and/or species of microorganism, the antibody will bind to more than one strain and/or species of microorganism.

In yet a further aspect, the invention provides compositions useful as vaccines wherein said compositions comprise a peptide of formula I. When more than one set of epitopic amino acids is used in the peptide of formula I and the sets of epitopic amino acids are from different strains and/or species of microorganism, the composition is useful to provide cross protection to more than one strain and/or species of microorganism.

In still a further aspect, the invention provides methods of preventing a microbial infection comprising administering to a mammal susceptible to said infection a peptide of formula I. When more than one set of epitopic amino acids is used in the peptide of formula I and the sets of epitopic amino acids are from different strains and/or species of microorganism, the composition is useful to prevent infection by several strains and/or species of microorganism.

In yet another aspect, the invention provides a method of treating or preventing microbial infection in an animal susceptible to or suffering from such infection, comprising administering to said animal an effective amount of an antibody to a microbial protein, wherein said antibody is produced by administering a peptide of formula I to an animal. In particular, such administration may be used to provide passive immunization and/or prevent or alleviate symptoms of infection in said animal. When more than one set of epitopic amino acids is used in the peptide of formula I and the sets of epitopic amino acids are from different strains and/or species of microorganism, the method may treat or prevent infection by several strains and/or species of microorganism.

In still another aspect, the invention provides a method of determining the presence of a particular microorganism in a sample comprising contacting the sample with an antibody to a peptide of formula I which peptide comprises epitopes from the particular microorganism, and determining whether said antibody binds to a component of said sample. The sample may be a biological sample. The method may be used to determine the causative agent of a microbial infection. When more than one set of epitopic amino acids is used in the peptide of formula I and the sets of epitopic amino acids are from different strains and/or species of microorganism, the method may be used to simultaneously detect the presence of several strains and/or species of microorganism in the sample.

In a further still aspect, the invention provides a method for providing multiple epitopes for exposure to the immune system of an animal comprising administering to the animal a peptide of formula I wherein said peptide epitopes are from different proteins. In a preferred embodiment, the peptide epitopes are from different proteins of a single microorganism or from different strains and/or species of microorganisms.

In still a further aspect, the invention provides a method for determining the presence of antibodies to a microbial protein in a biological sample comprising contacting the sample with a peptide of formula I which peptide comprises at least one epitope from the microbial protein and determining binding of antibodies in the sample to the peptide. The method may be used to determine whether an animal has been exposed to a microorganism.

### BRIEF DESCRIPTION OF THE DRAWINGS

FIG. 1 is a hypothetical representation of the pneumococcal surface depicting several antigens present on the surface. The coiled-coil regions from PspA and PspC are believed to project away from cell wall.

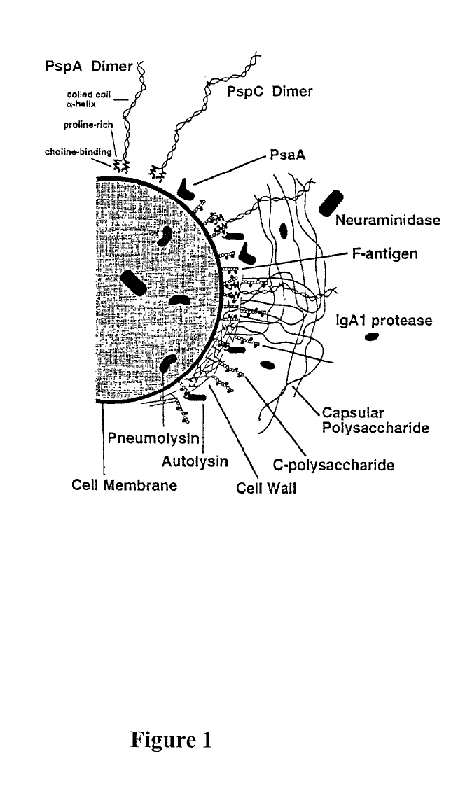

FIG. 2 is a diagrammatic representation of the PspA and PspC molecules highlighting their regions of homology. Each protein consists of 4 domains, a coiled-coil domain, a proline rich region, a choline binding region and a hydrophobic tail. High sequence homology exists in the region 2 sections of the coiled-coil domains between PspA and PspC.

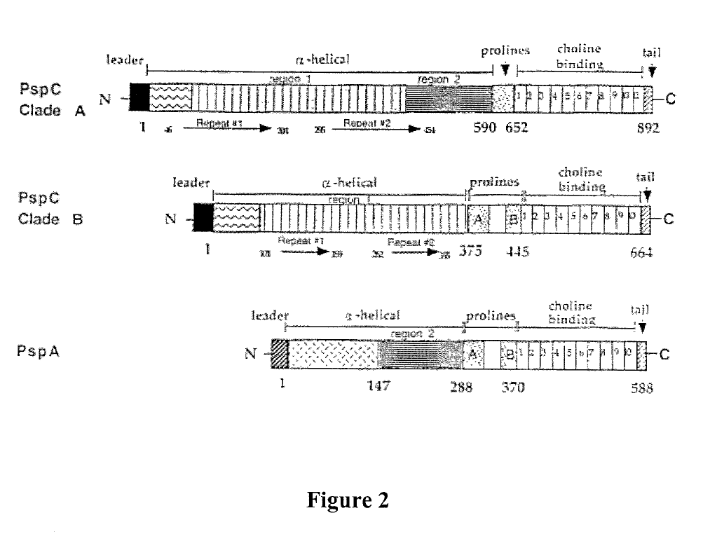

FIG. 3 depicts the cross-sectional view of a coiled-coil structure consisting of two peptide chains. The 7 positions of the heptad repeat in chain 1 are designated a, b, c, d, e, f and g, while the 7 positions of the heptad repeat in chain 2 are designated a′, b′, c′, d′, e′, f′, and g′. The open arrows indicate hydrophobic interactions between a and a′, and d and d′, respectively.

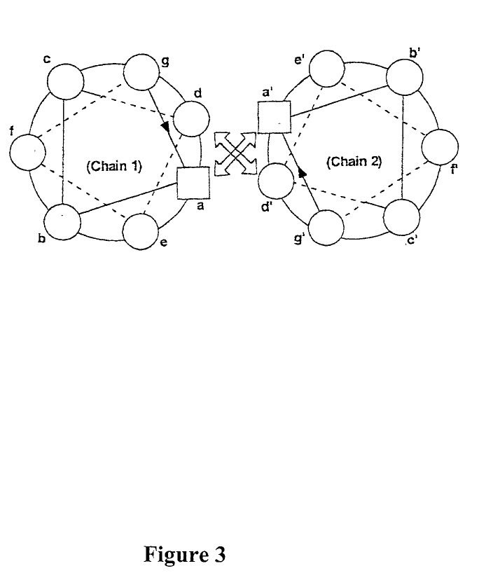

FIG. 4 shows the steps of combining the epitopes of several clinically relevant strains of S. pneumoniae into a coiled-coil template to make an immunogenic peptide.

FIG. 5 shows the CD spectrum of the peptide of FIG. 4 in the presence and absence of TFE.

FIG. 6 shows a region of PspA which is highly conserved. A consensus sequence which is selected such that it conforms with formula I is also shown.

FIG. 7 shows the CD spectrum of peptide CVX0270, which contains a portion of the consensus sequence shown in FIG. 6.

FIG. 8 shows the thermal denaturation profile of peptide CVX0270.

### DETAILED DESCRIPTION OF THE INVENTION

This invention relates to the use of a coiled-coil structural scaffold to generate structure-specific peptides, including synthetic peptides derived from naturally occurring protein antigens. The structure of the synthetic peptides utilizes a scaffold of heptad repeat units into which epitopes of native coiled-coil proteins are spliced.

In particular, the synthetic peptides are based on microbial proteins, especially surface proteins, which occur naturally in the coiled-coil form, such as pneumococcal surface proteins A and C. The synthetic peptides are immunogenic and can be used to elicit an immune response in an animal. Accordingly, they are useful as vaccines or to stimulate antibody production or cell-mediated immunity which recognizes the naturally occurring protein.

Prior to describing the invention in further detail, the terms used in this application are defined as follows unless otherwise indicated.

Definitions

A “native protein” is a protein which exists in nature.

A “coiled-coil protein” is a protein which, at least in part, forms a coiled-coil. A coiled-coil protein may have other conformational structures in addition to the coiled-coil. Encompassed within the term coiled-coil proteins are the proteins which have been shown to assume a coiled-coil structure and the proteins which are predicted to form coiled-coils by using a computer algorithm.

A “microbial protein” is a protein derived from a microorganism such as a bacterium, archaebacterium, fungus, virus, protozoan, parasite, alga, slime mold, or prion.

A “peptide” as used herein is a peptide or a protein. In other words, a peptide referred to in this application may contain any number of amino acids.

A “peptide of formula I” is a peptide which comprises a synthetic fragment, which fragment consists of formula I.

An “epitope” is a part of a protein or peptide which is an antigenic determinant.

A “derivative” of an amino acid is a non-naturally occurring amino acid residue or a chemically modified amino acid. Amino acid derivatives may be used to increase the half life of the peptide in serum or tissue, or to increase antigenicity of the peptide.

Non-naturally occurring amino acids may be but not limited to D-isomers, norleucine, 4-amino butyric acid, aminoisobutyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid and 2-thienyl alanine.

A chemically modified amino acid is an amino acid with a side chain modification. For example, the amino group of lysine may be modified by alkylation with an aldehyde followed by reduction with NaBH₄; amidation with methylacetimidate; acylation with acetic anhydride; carbamoylation with cyanate; trinitrobenzylation with 2,4,6,-trinitrobenzene sulphonic acid (TNBS); acylation with succinic anhydride and tetrahydrophthalic anhydride; and pyridoxylation with pridoxal-5′-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatization, for example, to a corresponding amide.

The sulfhydryl group may be modified by carboxymethylation with iodoacetic acid or iodoacetamide; performic acid oxidation to cysteic acid; formation of a mixed disulfide with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; or carbamoylation with cyanate at an alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues may be altered by nitration with tetranitromethane to form a 3-nitrotyrosine derivative.

Modification of the imidazole ring of a histidine residue may be accomplished by alkylation with iodoacetic acid derivatives or diethylpyrocarbonate.

A “peptide” may be a peptide containing naturally existing amino acids or amino acid derivatives. A peptide may also contain cross links between different portions of the peptide. For example, coiled-coil structures are often composed of two strands or three strands. It is preferable that the different strands of the coiled-coil are linked by disulfide bonds, which stabilize the coiled-coil structure. More preferably, the peptide contains a lactam bridge between the sidechains of lysine and glutamic acid which are spaced 3 or 4 residues apart (Houston et al., 1996). These lactam bridges are incorporated preferably at the N- and C-termini of the peptide sequence and not in regions of the sequence where the epitope is being displayed.

A “solvent exposed” or “solvent accessible” amino acid residue is an amino acid residue of a protein or peptide which is exposed to the solvent when the protein or peptide exists in a solution. The solution is preferably an aqueous solution, more preferably a physiologically compatible solution, such as blood, lymphatic fluid or the benign buffer. In particular, “solvent exposed” amino acid residues refer to the residues at the b, c, e, f or g positions of a native epitope which forms or is predicted to form a coiled-coil.

A benign buffer is a phosphate buffered solution. The “benign buffer” as used in this application is 50 mM KH₂PO₄, 50 mM KCl, pH 7.0.

An “antibody” is a protein molecule that reacts with a specific antigen and belongs to one of five distinct classes based on structural properties: IgA, IgD, IgE, IgG and IgM.

An “immune response” is the development in the host of a cellular and/or antibody-mediated immune response to a composition or vaccine of interest. Such a response may consist of the production of one or more of the following: antibodies, B cells, helper T cells, suppressor T cells, and/or cytotoxic T cells directed specifically to an antigen or antigens included in the composition or vaccine of interest.

A “vaccine” is a molecule which is capable of eliciting in an animal an immune response which prevents, partially or completely, infection by a pathogen.

“Elicit” or “stimulate” an immune response is to cause an immune response by exposing an immune system to an immunogen.

An “immunogen” is a molecule which is capable of eliciting an immune response in an animal.

“Prevent a microbial infection” is to prevent, completely or partially, the development of a microbial infection.

“Treat a microbial infection” is to reduce, completely or partially, the symptoms of a microbial infection after the onset of the microbial infection.

A “sample” is an aliquot or a representative portion of a substance, material, or population. For example, a sample may be a sample of water, sewage, oil, sand, blood, biological tissue, urine or feces.

A “biological sample” is a sample collected from a biological subject, such as an animal, plant or microorganism.

An “effective amount” is an amount which is sufficient to achieve the intended purposes. For example, an effective amount of a vaccine is an amount of the vaccine sufficient to elicit an immune response in the recipient of the vaccine to protect the recipient from contracting the target disease or to prevent or alleviate medical conditions associated with the disease.

The Synthetic Coiled-Coil Peptide

It has been shown previously that PspA is highly variable among different strains of S. pneumoniae. Mapping studies using monoclonal antibodies raised against PspA indicate that the major cross-reactive epitopes are found in the last 100 amino acids of the coiled-coil domain (residues 192-270 of SEQ ID NO: 1) (McDaniel et al., 1994). Based on the sequence of this region, it has been possible to group PspA proteins into 6 clades, which comprise three families. Animal studies have shown that immunization with PspA elicits an immune response which is not only cross-reactive to other strains in the same PspA family, but that this cross-reactivity extends between families (Briles, et al. 1999; McDaniel et al., 1991; Tart et al., 1996). In addition, immunization with purified PspC fragments generates antibodies that are protective against sepsis and cross react with PspA (Brooks-Walter et al., 1999). The cross-protection results suggest that despite the variation among the coiled-coil domain for PspA and PspC, there must be conserved epitopes.

The first evidence that immunity to PspA might be protective was the finding that PspA specific monoclonal antibodies (MAbs) protected mice from fatal sepsis. Subsequently, it was shown that passive protection with IgG or IgM monoclonal antibodies to PspA from strain R×1 protected mice from death following ip or iv challenge with 10⁵ times the LD₅₀ of S. pneumoniae strain WU2 (Briles, et al. 1989). The MAbs were shown to clear the pneumococci from the blood of the challenged mice by a complement-mediated mechanism. Active immunization studies utilized a 27 kDa fragment of PspA from S. pneumoniae strain R×1 (residues 1-245 of SEQ ID NO: 1) which contain most of the helical domain of the protein. Immunization of CBA/N mice with 5 μg of this fragment in combination with the adjuvant FCA followed by a booster dose in PBS were protected from an iv challenge of 300 cfu (30 times LD₅₀) of strain WU2 (Talkington et al., 1991). In more recent studies when mice received 1 or 5 μg of a fragment comprising residues 1-303 with no adjuvant, it elicited protection against fatal infection following iv challenge with 480 cfu of the more virulent strain A66.1 (Briles et al. 1998). Passive protection studies in mice with immune sera from immunized mice or rabbits can protect against fatal sepsis from challenge doses of 10 to 100 times LD₅₀ of the challenge strain.

While PspA can be divided into numerous serological types, it has been shown to be a highly cross-reactive protein. Sera from a single rabbit immunized with the helical portion of PspA from strain R×1 can recognize PspA from all pneumococci in Western blots. When CBA/N mice were challenged with 14 different S. pneumoniae strains (˜200×LD₅₀ dose) following immunization with PspA from strains D30, WU2 and BG9739, each PspA exhiybited cross-protection to most challenge strains. The helical domain of PspA from the strain R×1 (residues 1-314 of SEQ ID NO: 1) was recently evaluated for safety and immunogenicity in human volunteers (Nabors et al., 2000). When patients were immunized with 5 to 125 mg of this fragment absorbed onto aluminum hydroxide, high levels of circulating antibodies to the R×1 fragment were elicited as well as antibodies reactive to heterologous PspA molecules.

The PspA sequence from S. pneumoniae strain R×1 was analyzed using the Peptools program from Biotools Inc. The Peptools structure prediction algorithm is based on identifying known protein-folding motifs combined with consensus prediction based on the results from four prediction algorithms. The Peptools program also predicts a highly helical region that spans over 300 amino acids for the N-terminal portion of PspA. Peptools program is also capable of predicting which sites of proteins are antigenic by measuring hydrophilicity, solvent accessibility, flexibility, and turn propensity of various sequences, and in addition, matches sequences against a database of known B-cell epitopes. Peptools identified a number of potential B-cell epitopes in the coiled-coil domain which are listed in Appendix 1, which is incorporated herein by reference in its entirety.

The folding of PspA and PspC (or other proteins which exist as coiled-coils) into a coiled-coil results in certain residues being solvent accessible and certain residues which form the hydrophobic core being solvent inaccessible. The solvent accessible amino acids are located at positions b, c, e, f and g of the heptad repeat while amino acids a and d are buried. Thus, residues at solvent accessible positions must be responsible for mediating an immune response, e.g., antibody production. The epitopes formed by the coiled-coil region must be discontinuous since the a and d residues are not exposed. Thus, the epitopes from these proteins must be derived from the solvent accessible positions b, c, e, f and g.

We discovered that the stability of the hydrophobic core of a coiled-coil protein may be maximized by incorporation of Ile residues at a positions and Leu residues at d positions of the heptad repeat. Accordingly, the present invention provides a synthetic peptide comprising formula I:

| | |
|---|---|
| (AXXDXXX)ₙ | (SEQ ID NO:15) |

Wherein

A is Ile or a derivative thereof;

D is Leu or a derivative thereof;

each X is an amino acid residue or derivative thereof which corresponds to an amino acid residue of an epitope of a native coiled-coil protein;

the X residues in each (AXXDXXX) repeat form a set of X residues; and

n is equal to or greater than 1.

While the preferred embodiment has Ile at the a position and Leu at the d position, also contemplated in the present invention are peptides containing Val, Ile or Leu at the a or d positions. Thus, the amino acid at the a position may be Val, Ile or Leu, and the amino acid at the d position may, independently, also be Val, Ile or Leu.

Although the coiled regions of PspA and PspC are quite long, synthetic coiled-coil forming sequences may be shorter. It has been shown that if lactam bridges are included, a synthetic coiled-coil may be as short as 14 residues (Houston et al., 1996). In the absence of lactam bridges, we have found that helical peptides containing 21 amino acids were stable. In addition, linking of the individual helices together by an intermolecular disulfide bond further stabilizes these structures. The small coiled-coils can serve as scaffolds into which epitopes from native coiled-coil proteins, such as PspA and PspC, are spliced. A potential scaffold has the following sequence:

| | |
|---|---|
| Cn1eG-(IXXLXXX)ₙ | (SEQ ID NO:16) |

Wherein Nle is norleucine, an isomer of leucine containing an unbranched side chain. Residues that make up epitopes from native coiled-coil proteins such as PspA and PspC (residues b, c, e, f and g) would be inserted into the X positions. The epitopes could potentially be determined by using algorithms and bioinformatics programs such as Peptools. Epitopes could also be mapped by monoclonal antibodies or by analysis of polyclonal antisera raised against native proteins on a Biacore instrument. Epitopes from different native proteins such as from different strains of S. pneumoniae could be spliced together to yield novel sequences. The peptide may be synthesized by peptide synthesis methods established in the art, or an expression vector encoding the peptide sequence of interest can be constructed and used to express the peptide. The I and L residues in this scaffold may, independently, be replaced by any amino acid selected from I, L or V.

The amino acids at the b, c, e, f, and g positions in the present invention are the solvent exposed amino acids from natural coiled-coils of the particular protein of interest. Therefore, the present invention is distinct from WO 01/00010, which is directed to displaying solvent exposed amino acids from a non-coiled-coil protein in a coiled-coil template. As a result, a non-coiled-coil epitope is converted to a coiled-coil conformation, and it is possible that antibodies raised against the coiled-coil conformation will not recognize the original, non-coiled-coil epitope. In contrast, a peptide made according to formula I of the present invention contains at the b, c, e, f, and g positions the amino acids from the b, c, e, f, and g positions of a coiled-coil protein. The resulting peptide, while being more immunogenic, will elicit the same immune response as the original epitope with respect to specificity.

The present invention is also distinct from WO 96/11944, which is directed to embedding one peptide in another. Briefly, WO 96/11944 discloses that one can enhance the immunogenicity of a peptide by flanking the peptide with amino acids which have a strong propensity to form the same conformation as the peptide of interest, thereby stabilizing the conformation of the peptide. For example, a peptide which may form a coiled-coil can be embedded in the amino acid sequences of GCN4, which forms relatively stable coiled-coils. The resulting chimeric peptide had a high helical content when dissolved in the α-helix inducing solvent trifluoroethanol, but its helical content under aqueous conditions was low. The present invention, on the other hand, does not embed a peptide fragment in another amino acid sequence. Instead, the amino acids at the a and d positions of the peptide are specifically chosen and replaced where appropriate in order to increase the stability of the coiled-coil conformation, and the resulting peptide is typically helical even in aqueous solutions.

The following example demonstrates the basic features of the present invention. As mentioned previously, the C-terminal portion of the coiled-coil domain from the R×1 PspA protein has been shown to contain a number of cross-protective epitopes. The Biotools software identified a number of such epitopes including residues 153-170 and residues 181-198 that will be used to illustrate the construction of a coiled-coil immunogen (Example 1). The a and d position of these epitopes are highlighted in bold text while the solvent accessible amino acids are in italics. These positions are replaced by Ile at a positions and Leu at d positions of the template. By simply splicing out the solvent exposed residues (italicized) from the potential epitopes and incorporating them into the scaffold, one is able to create unique polypeptide sequences which are immunogenic. The coiled-coils described in Example 1 will have approximate molecular weights of 7,000 Da and may be large enough to elicit an immune response on their own. Alternatively, these molecules could be coupled to carrier proteins such as tetanus toxoid or KLH, or attached to liposomes using techniques known to those of skill in the art.

It must be stressed that the residues must be kept in phase such that, for example, a c residue from the protein must correspond to an c residue in formula I. Residues from either of the two adjoining sequences can be used to fill any gaps that might arise during the splicing. Since the coiled-coil is composed of two α-helices, the two helical strands can bear different sequences, thus increasing the number of epitopes in the entire molecule. Through incorporation of a number of epitopes from different clades, the coiled-coil peptides will cross react among different clades.

By maintaining a constant hydrophobic core, splicing in only solvent exposed residues and mixing epitopes from different proteins, unique sequences are produced. Thus, a formula I peptide may have the amino acids from the b, c, e, f and g positions of PspA in one heptad repeat and those from PspC in another repeat. When this peptide is used as a vaccine, immunity against both PspA and PspC is induced, thereby providing double protection against S. pneumoniae.

A particularly appealing approach is to include amino acids from both the surface proteins (such as PspA and PspC) and the surface adhesin protein (PsaA). The adhesin protein is responsible for pathogenesis of S. pneumoniae in the nasopharyngeal area while the surface proteins are involved in systemic symptoms. It has been shown that PsaA may be an effective vaccine to prevent nasopharygeal carriage while PspA is a better vaccine candidate for systemic infections (Briles et al., 2000). Therefore, a peptide comprising epitopes from both PsaA and PspA will provide a defense against both the nasopharygeal and systemic infections by the bacteria. This can be achieved by splicing the solvent exposed amino acids from the α-helical region of PsaA into one heptad repeat of a Formula I peptide, and the solvent exposed amino acids from the α-helical region of PspA into another heptad repeat of the same peptide. Similarly, a hybrid peptide comprising both PspC epitopes and PsaA epitopes can be prepared.

Alternatively, the formula I peptide may contain the solvent exposed amino acids of proteins from different strains of a microorganism or from different microorganisms, and the resulting vaccine can be effective against all the strains or microorganisms used. Similarly, a consensus sequence may be deduced for a certain protein from different strains and used as a vaccine against all these strains (see Example 3).

In order to improve the helical stability of a peptide, the e and g positions of the heptad repeat of formula I may contain glutamic acid and lysine. These residues form salt bridges between e of one strand and g′ of the other strand, as well as g of one strand and e′ of the other strand. These salt bridges further help to stabilize the coiled-coil conformation. To stabilize individual α-helices, lysine residues and glutamic acid residues can be introduced at positions 3-4 residues apart to facilitate the formation of an intrastrand salt bridge between their sidechains. One can also increase the stability of the coiled-coil by the inclusion of amino acids with high helical propensity such as alanine or aminoisobutyric acid.

Alternatively, a stabilizing strand may be used which does not contain epitopes. The sole purpose of the stabilizing strand is to increase the helical content of the strand which contains the epitopes. A stabilizing strand is capable of forming a very stable helix which supports and stabilizes the helical structure formation of the epitope-containing strand. Preferably, the epitope has lysine and/or glutamic acid at the e or g positions, and the stabilizing strand can provide an amino acid with the opposite charge at the e′ or g′ positions to form an interstrand salt bridge. An example of a stabilizing strand is:

| | |
|---|---|
| CN1eGGG(EIEALKK)₅ | (SEQ ID NO:2) |

The structural and conformational integrity of these peptides can be readily determined by circular dichroism (CD) spectroscopy. The peptides useful in the present invention have helical characteristics in aqueous solutions (see Examples 2 and 3). Therefore, the native epitopes can be correctly presented in a physiological environment. In contrast, previously reported synthetic coiled-coil proteins do not form helices in aqueous solutions (WO 96/11944). Peptides useful in the present invention have a helical content of at least 20% in benign buffer as determined by CD spectroscopy. The helical content is preferably at least 40%, more preferably at least 60% and most preferably at least 80% in benign buffer.

The stability of the peptides may be determined by thermal denaturation assays or chemical denaturation assays such as the guanidine chloride denaturation assay. Such assays are known to those skilled in the art. The stability of the peptides may also be indirectly assessed by inhibition ELISA, wherein the ability of a peptide to inhibit the interaction between an antigen and an antibody serves as an indicator of how stable the peptide is in assuming the necessary conformation to compete with the antigen for the antibody.

Epitopes from other microbial coiled-coil proteins may also be used in the claimed invention. In particular, epitopes from microbial surface proteins which contain coiled-coil structures may be used. Examples of microorganisms whose proteins are useful in the claimed invention include, but are not limited to, Haemophilus influenza, and particularly its hypothetical protein H10500; the Lyme disease spirochete, i.e., Borrelia burgdorferi, in particular its surface lipoprotein P27; Legionella, especially its hypothetical protein ppIB; E. coli, particularly its conserved YigN protein; Neisseria spp, and in particular Neisseria meningitidis and its putative periplasmic protein; Moraxella catarrhalis, especially its proteins UspA1 and UspA2; Mycoplasma spp, and in particular, Mycoplasma pneumoniae and its hypothetical protein EO7; Chlamydia spp, particularly hypothetical protein CT825; Plasmodium falciparum, and particularly its hypothetical protein PFB01450 and mature-parasite-infected erythrocyte surface antigen; Trypanosoma spp; Staphylococcus aureus; Streptococcus, particularly Group A and Group B; Bordetella pertussis; Salmonella; Streptococcus mutans; Cryptococcus neoforms; Klebsiella pneumonia; Pseudomonas aeruginosa; viruses; fungi; and the like.

Moreover, other coiled-coil proteins which do not come from a microbial origin may be used in the same fashion. For example, it is common to raise antibodies to a protein for therapeutic purposes or to study the functions of this protein. However, not every protein of interest is sufficiently immunogenic, and it may be difficult to raise antibodies specifically directed to particular coiled-coil regions of the native protein. The present invention may be used to optimize antibody preparation for any coiled-coil protein no matter what origin, function or subcellular localization this protein may have. Moreover, the present invention also enables production of antibodies directed to particular coiled-coil regions, by including only the region of interest in the peptide used to raise antibodies.

Examples of non-microbial coiled-coil proteins include, without being limited to, estrogen receptor binding fragment associated gene 9 (Nakashima et al., 1999), leukemia associated protein 5 (Kapanadze et al., 1998), uveal autoantigen (Yamada et al., 2001), angiopoietin precursor (Davis et a., 1996), NF-kappa B essential modulator (Li et al., 1999), Tumor susceptibility gene 101 protein (Li et al., 1997), cytosolic ovarian carcinoma antigen 1 (Chang et al., 1994), TACC 1 (Still, 1999), and TPR (Miranda et al., 1994).

In addition to the microbial and non-microbial proteins listed herein, a skilled artisan can identify other coiled-coil proteins according to established methods in the art. For example, if the amino acid sequence of a protein is available, various computer algorithms such as Multicoil or Peptool can be used to predict if the protein forms a coiled-coil. For a protein which is substantially pure, CD spectroscopy can be used to determine its actual structure as described herein.

The Compositions

A composition comprising the coiled-coil peptides of formula I can be used to elicit an immune response in an animal for at least two purposes. Where the composition acts as a vaccine by eliciting an immune response in the animal, the resulting antibodies or T-cell mediated immunity can protect the animal from a subsequent attack involving the same epitopes (active immunity). Alternatively, the composition can be used to produce antibodies which can be used as a research tool, or administered to a second animal to protect the second animal from a subsequent attack involving the same epitopes (passive immunity).

To augment the immune response elicited, it may be preferable to couple the peptides of formula I, especially the smaller peptides (e.g., those containing one to four heptad repeats), to a carrier protein.

In addition, the coiled-coil peptides of formula I or their conjugates with carrier proteins may be further mixed with adjuvants to elicit an immune response, as adjuvants may increase immunoprotective antibody titers or cell mediated immunity response. Such adjuvants may include, but are not limited to, Freunds complete adjuvant, Freunds incomplete adjuvant, aluminum hydroxide, dimethyldioctadecyl-ammonium bromide, Adjuvax (Alpha-Beta Techlnology), Inject Alum (Pierce), Monophosphoryl Lipid A (Ribi Immunochem Research), MPL+TDM (Ribi Immunochem Research), Titermax (CytRx), QS21, the CpG sequences (Singh et al., 1999), toxins, toxoids, glycoproteins, lipids, glycolipids, bacterial cell walls, subunits (bacterial or viral), carbohydrate moieties (mono-, di-, tri-, tetra-, oligo- and polysaccharide) , various liposome formulations or saponins. Combinations of various adjuvants may be used with the antigen to prepare the immunogen formulation.

The composition may be administered by various delivery methods including intravascularly, intraperitoneally, intramuscularly, intradermally, subcutaneously, orally, nasally or by inhalation. The composition may further comprise a pharmaceutically acceptable exicipient and/or carrier. Such compositions are useful for immunizing any animal which is capable of initiating an immune response, such as primate, rodent, bovine, ovine, caprine, equine, leporine, porcine, canine and avian species. Both domestic and wild animals may be immunized. The exact formulation of the compositions will depend on the particular peptide or peptide-carrier conjugate, the species to be immunized, and the route of administration.

The antibodies produced against a coiled-coil protein can be included in a pharmaceutical composition and administered to an animal. The pharmaceutical composition typically comprises a pharmaceutically acceptable carrier, and may include pharmaceutically acceptable excipients. The pharmaceutical composition can be administered intravascularly, intraperitoneally, intramuscularly, intradermally, subcutaneously, orally, nasally or by aerosol inhalation. Preferably the pharmaceutical composition is administered intravascularly, intramuscularly, nasally or by aerosol inhalation.

Also encompassed by the present invention are antibodies, particularly monoclonal antibodies, which are derived from the antibodies produced against a peptide of formula I. In particular, hybridomas can be generated using a peptide of formula I, and recombinant derivative antibodies can be made using these hybridomas according to well-known genetic engineering methods (for a review, see Winter et al., 1991). For example, the DNA fragment coding for the variable regions of the monoclonal antibodies can be obtained by polymerase chain reactions (PCR). The PCR primers can be oligonucleotides which are complementary to the constant regions of the heavy chain or light chain, and the PCR template can be the total cDNA or genomic DNA prepared from the hybridomas. Alternatively, a cDNA library can be prepared from the hybridomas and screened with probes which correspond to the constant regions of immunoglobulin heavy chain or light chain to obtain clones of the heavy chain or light chain produced by the particular hybridoma.

Subsequently, the DNA fragment for the variable regions can be inserted into an expression vector and joined in frame with the cDNA sequences of a selected constant region. The constant region can be the human constant sequences to make humanized antibodies, the goat constant sequences to make goat antibodies, the IgE constant sequences to make IgE which recognizes the peptide of formula I, and the like. Thus, antibodies with the same antigen recognition ability but different constant regions can be produced. Of particular interest are humanized antibodies, which can be used as therapeutic agents against a disease associated with the cognate antigen in humans without eliciting an undesired immune response against the humanized constant region.

Other methods known in the art to humanize antibodies or produce human antibodies can be utilized as well, including but not limited to the xenomouse technology developed by Abgenix Inc. (U.S. Pat. Nos. 6,075,181; 6,150,584) and the methods developed by Biovation, Bioinvent Internatioal AB, Protein Design Labs., Applied Molecular Evolution, Inc., ImmGenics Pharmaceuticals Inc., Medarex, Inc., Cambridge Antibody Technology, Elan, Eos Biotechnology, MedImmune, MorphoSys or UroGensys Inc. Likewise, other methods known in the art to screen human antibody secreting cells to coiled-coil peptide antigens can also be utilized.

The formulation for the composition, comprising either a coiled-coil peptide or an antibody against a coiled-coil peptide, will vary depending on factors such as the administration route, the size and species of the animal to be administered, and the purpose of the administration. Suitable formulations for use in the present invention can be found in Remington's Pharmaceutical Sciences.

The following examples are offered to illustrate this invention and are not to be construed in any way as limiting the scope of the present invention.

### EXAMPLES

In the examples below, the following abbreviations have the following meanings. Abbreviations not defined have their generally accepted meanings.

° C. = degree Celsius
hr = hour
min = minute
μM = micromolar
mM = millimolar
M = molar
ml = milliliter
μl = microliter
mg = milligram
μg = microgram
rpm = revolutions per minute
ID = inner diameter
TFE = trifluoroethanol
EDT = ethane dithiol
TFA = trifloroacetic acid
PBS = phosphate buffered saline
β-ME = β-mercaptoethanol
DMSO = dimethylsulfoxide
Nle = norleucine
ELISA = enzyme linked immunosorbent assay
HRP = horse radish peroxidase
TD = thymus dependent
TT = tetanus toxoid
KLH = keyhole limpet hemocyanin
BSA = bovine serum albumin

The one letter code and the three letter code for amino acids used throughout this application are listed below:

A = Ala = Alanine
C = Cys = Cysteine
D = Asp = Aspartic Acid
E = Glu = Glutamic Acid
F = Phe = Phenylalanine
G = Gly = Glycine
H = His = Histidine
I = Ile = Isoleucine
K = Lys = Lysine
L = Leu = Leucine
M = Met = Methionine
N = Asn = Asparagine
P = Pro = Proline
Q = Gln = Glutamine
R = Arg = Arginine
S = Ser = Serine
T = Thr = Threonine
V = Val = Valine
W = Try = Tryptophan
Y = Tyr = Tyrosine

### MATERIALS AND METHODS

Peptide Synthesis and Purification

The peptide analogs were prepared by t-Boc chemistry on an Applied Biosystem 431 A peptide synthesizer. The peptide resin (700 mg) was cleaved with 10 mL of HF containing 10% anisole and 2.5% EDT for 1 h at 0-4° C. After removal of the HF the peptide/resin was transferred to a sintered glass funnel and washed with diethyl ether (2×50 mL) followed by glacial acetic acid (2×50 mL). The acetic acid solution was then lyophilized. The crude peptide was taken up in 15 mL of water containing 0.05% TFA and 3 mL acetic acid. After stirring and sonication, the mixture was transferred to 1.5 mL Eppendorf tubes and centrifuged at 13000 rpm. The supernatant was collected and filtered through a Millex GV 0.22 μm syringe filter. This solution was loaded onto a Zorbax RX-C8 (22.1 mm ID×250 mm, 5 μm particle size) through a 5 mL injection loop at a flow rate of 3 mL/min. The purification was accomplished by running a linear AB gradient of 0. 1% B/min where solvent A is 0.05% TFA in water and solvent B is 0.05% TFA in acetonitrile.

A linker and cysteine residue was incorporated to enable the strands of the coiled-coil to be linked together by a disulfide bond. The disulfide-bridged coiled-coils were formed by overnight air oxidation at room temperature of ˜10 mg/mL peptide in 100 mM NH₄HC0₃, pH 8.5. All peptides were characterized by analytical HPLC and electrospray mass spectrometry, and protein concentrations as well as amino acid composition were determined by amino acid analysis. Peptides were conjugated to the carrier protein tetanus toxoid rising the photoreactive reagent benzoylbenzoic acid which was coupled to the N-terminus of the peptide by conventional solid-phase chemistry described above. This photoaffinity probe is stable under the conditions of t-Boc chemistry. A norleucine residue is incorporated into the linker region of the peptide to enable the quantitation of the peptide attached to the carrier protein by means of amino acid analysis. To 10 mg of tetanus toxoid dissolved in 100 mm of NH₄HCO₃ pH 8.3 buffer was added 6 mg of peptide and the solution was irradiated for 1 h at a wavelength of 350 mn. To remove any unreacted peptide, the solution was transferred to a 15 mL dialysis cassette and dialyzed against 4 L of 20 mM NaH₂PO₄pH 7.0 buffer. HPLC analysis of the dialysate was used to determine the amount of unreacted peptide present in the irradiated solution. The dialysis was deemed to be complete when residual peptide was less than 1% of the total area under the curve in the HPLC chromatogram.

CD Spectroscopy

Circular dichroism (CD) spectroscopy was performed using a Jasco J-500C spectropolarimeter (Jasco, Easton, Md.) equipped with a Jasco DP-500N data processor. A 10-fold dilution of an ˜500 mM stock solution of the disulfide-linked peptide was loaded into a 0.02 cm fused silica cell and ellipticity scanned from 190 to 250 nm. Each disulfide-bridged analog was analyzed by CD spectroscopy under benign conditions (50 mM phosphate, 100 mM KCI, pH 7.0) and also in the presence of 50% TFE in the same buffer. A Lauda water bath (model RMS, Brinkmann Instruments, Rexdale, Ont.) was used to control the temperature of the cell. CD spectra were the average of four scans obtained by collecting data at 0.1 nm intervals from 250 to 190 nm.

HPLC

Analytical HPLC was performed on a Beckman System Gold HPLC equipped with a diode array detector. Analyses were performed on a Zorbax SB-C8 column (4.5 mm ID×150, 3.5 μm particle size). Mobile phases were A: deionized purified water (18 MΩ/cm) containing 0.05% trifluoroacetic acid and B: acetonitrile (HPLC grade, 99.99%) containing 0.05% TFA. HPLC operating conditions are listed below:

| Flow rate | 1 mL/min |
|---|---|
| Injection Volume | 20 mL |
| Detector Wavelength | 215 nm |
| Gradient | 2.5% B/min |
| Total run time | 20 min |
| Integration Start Time | 5 min |
| Minimum Area (integration) | 25000 |

### Example 1

### Construction of a Synthetic Pneumococcal Immunogen

This Example illustrates the method of splicing the amino acid residues at the b, c, e, f or g positions of the coiled-coil structures of PspA into a synthetic immunogen. The Biotools software identified a number of coiled-coil motifs in PspA, including residues 153-170 and 181-198. As shown below, the a and d positions of these motifs are indicated in bold text while the solvent accessible amino acids are italicized. A space is inserted between the heptad repeats for clarity.

| | |
|---|---|
| LEEAEKK ATEAKQK VDA | residues 153-168 of SEQ ID |
| | NO:1 |
| AELENQ VHRLEQE LKEIDES | residues 181-198 of SEQ ID |
| | NO:1 |

The solvent exposed residues are incorporated into the coiled-coil template with isoleucine and leucine at the a and d positions, respectively:

```
            LEEAEKK ATEAKQK VDA 153-168
                                                  AELENQ VHRLEQE LKEIDES 181-198
                                                          +
      CN1eG-IXXLXXX IXXLXXX IXXLXXX IXXLXXX IXXLXXX
                                      coiled-coil template
                                                  −
CN1eG IEELEKK ITELKQK I--LENQ IHRLEQE IKELDES
                                                  −
                                                            fill gap with aa from either sequence
CN1eG IEELEKK ITELKQK IDALENQ IHRLEQE IKELDES
                                    (test peptide; SEQ ID NO:3)
```

The peptide is synthesized as described in Materials and Methods. Also synthesized is a control peptide containing the native sequence instead of isoleucine and leucine at the a and d positions:

| | |
|---|---|
| CN1eG LEEAEKK ATEAKQK VDALENQ VHRLEQE LKEIDES | (native control; SEQ ID NO:4) |

The CD profile and helical stability of the peptides are determined as described in Materials and Methods. The results show that the test peptide exists in helical form even in benign buffer (50 mM KH₂PO₄, 50 mM KCL, pH 7.0) in the absence of trifluoroethanol (TFE). By contrast, the native control needs the presence of TFE to form a helical structure. Similarly, the test peptide is much more stable than the native control when tested in a thermal denaturation experiment.

### Example 2

### A Synthetic Pneumococcal PspA Peptide

A synthetic peptide was prepared based on epitopes from three different Pneumococcal surface proteins A (PspA). The three PspA proteins and their capsular serotype are as follows:

| PspA Strain | Capsular Serotype |
|---|---|
| BG8743 | 23F |
| EF5668 | 4 |
| BG8090 | 19F |

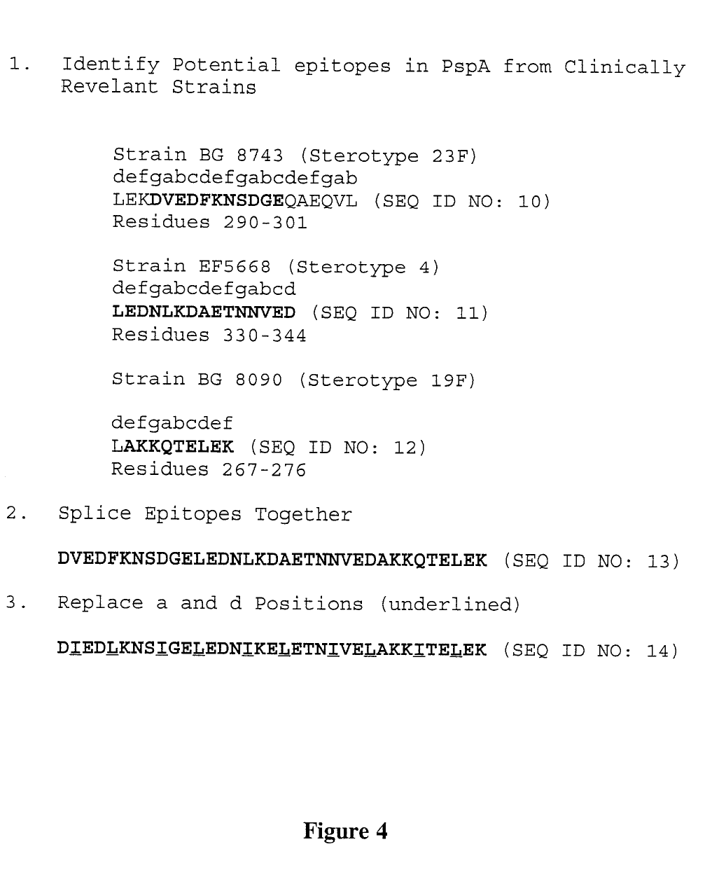

These proteins were chosen because of the prevalence of these serotypes in pneumococcal infections (Kalin, 1998). B-cell epitopes, shown in FIG. 4, were identified using the bioinformatics software Peptools (Edmonton, Alberta). The three epitopes were spliced together such that the coiled-coil repeat remained in register. A linker and cysteine residue was incorporated to enable the strands of the coiled-coil to be linked together by a disulfide bond.

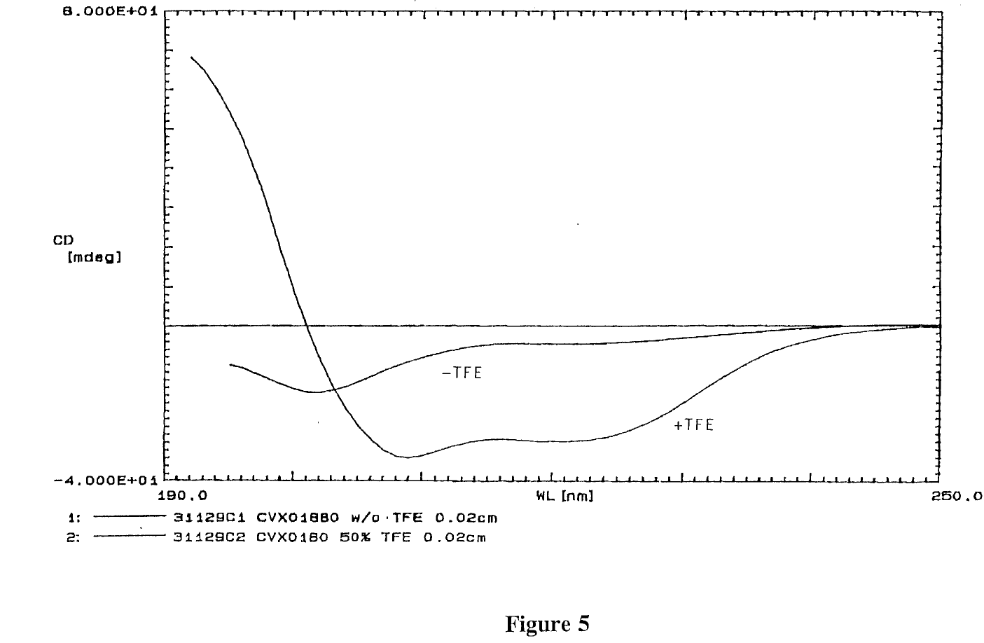

The CD spectrum of the peptide is shown in FIG. 5. In benign buffer (50 mM KH2PO4, 50 mM KCl, pH 7.0) the peptide adopts a conformation that is a mix of random coil and alpha helix. In the presence of 50% trifluoroethanol (TFE, which induces helical structure in peptides that have the propensity to fold into an alpha helix), the peptide adopts a highly helical conformation. The helical content in benign buffer is 17% of that in 50% TFE, indicating that the peptide forms helices in a physiologically compatible solution without TFE. Therefore, the peptide may be used to elicit an immune response under physiological conditions.

### Example 3

### Generation of a Consensus Coiled-Coil Sequence

To date 40 partial and full sequences of pneumococcal surface protein A from various strains have been released to various sequence databases. These proteins share a common architecture consisting of a leader sequence, a coiled-coil domain, a proline domain and a choline binding domain. Analysis of PspA sequences by the program MultiCoil (Wolf et al., 1997) indicates that the coiled-coil regions are broken up by sections which contain proline residues. Multiple sequence analysis using the software Biotools indicates that the published sequences vary in similarity from 24.1% to 97%. However, certain regions of the helical domain of these proteins are remarkably similar, suggesting that these conserved regions are important to the function of the protein

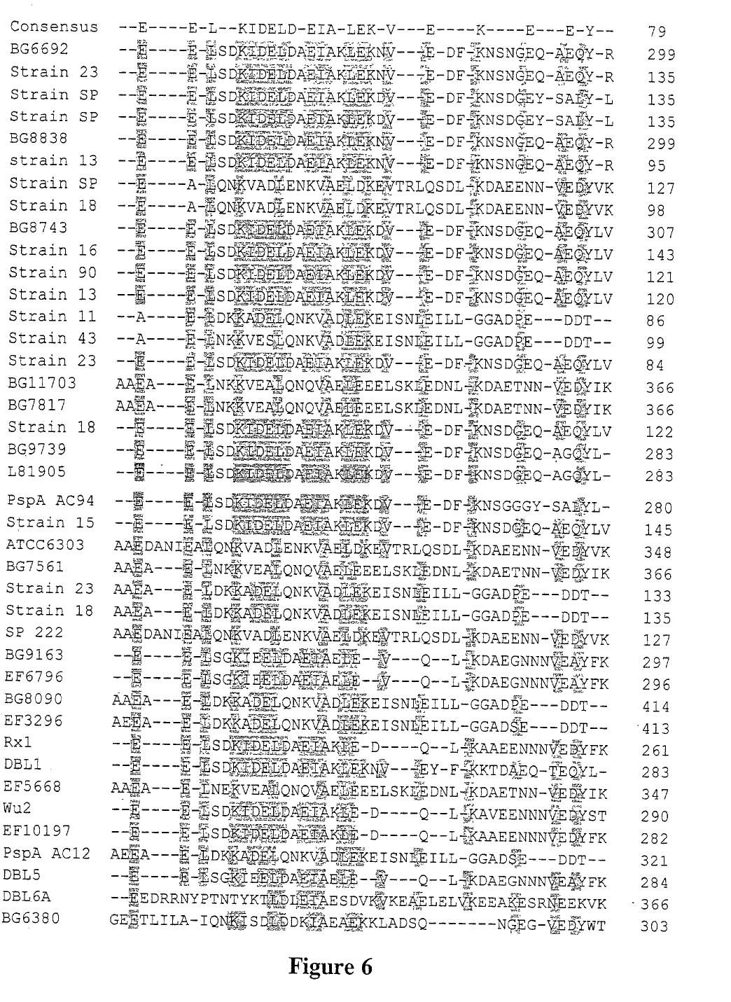

A consensus sequence (FIG. 6) was deduced from a conserved region which was shown to have antigenic activities. Complete and partial PspA sequences were downloaded from the National Library of Medicine PubMed web site into the bioinformatics software Peptools (Biotools, Edmonton, Alberta). A total of 40 complete and partial sequences were transferred to the alignment module of the software and a consensus threshold was set to 65%. The consensus threshold defines the minimum residue plurality amongst a group of aligned sequences. In effect, the consensus threshold acts to filter out insignificant matches and highlights conserved residues within a sequence. This allows for easy identification of similarities by visual inspection.

The last 100 amino acids of the coiled-coil region were the focus of sequence similarities since this region has been shown previously to harbor cross reactive epitopes. FIG. 6 illustrates the sequence alignment for residues found within this region. In particular, the alignment yielded a consensus sequence of EELX₁X₂KIDELDX₃EIAX₄LEKX₅ (SEQ ID NO: 5), in which the putative a and d positions were selected to be isoleucine and leucine, respectively. Preferably, X₁ is S, Q,N or D; X₂ is D, N or K; X₃ is A or N; X₄ is K, E or D; and X₅ is N, D or E.

A sequence containing the first section of this consensus region, EELSDKIDELD (SEQ ID NO: 6), was selected for antigenicity studies. The Ser and Asp residues were included into this sequence since these residues appear in the majority of sequences aligned.

A synthetic peptide containing EELSDKIDELD (SEQ ID NO: 6) was made according to formula I of the present invention. Thus, an isoleucine was inserted at the N-terminus of the above sequence according to formula I and flanking sequences were added to increase the length of the peptide. The complete sequence of this peptide, CVX0270, is as follows, wherein the residues from the consensus sequence are highlighted in bold script:

Acetyl-CN1eGEIEALKKKIEELSDKIDELEKEIK-amide (SEQ ID
NO:7)

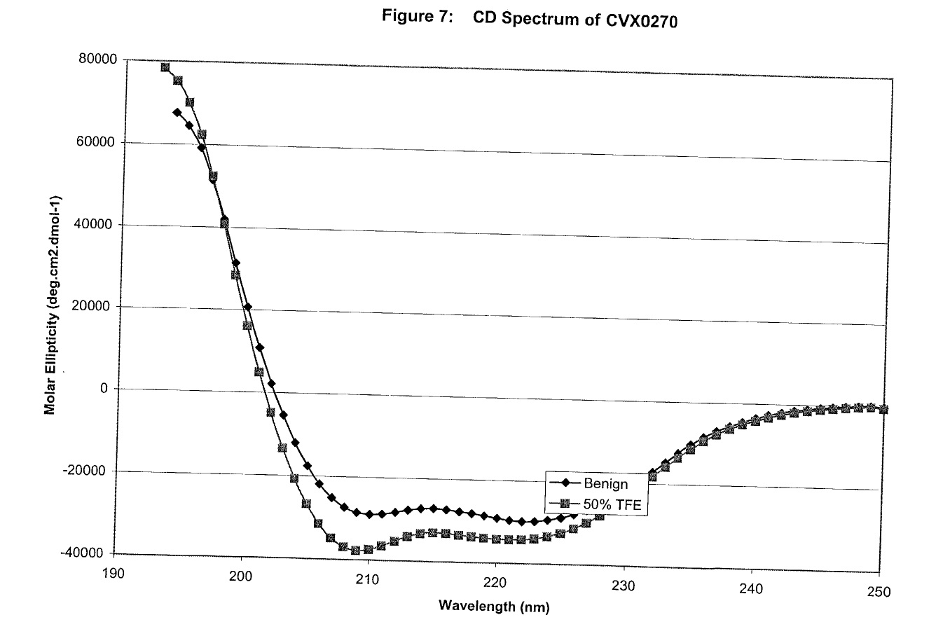

The CD spectra were performed under benign conditions (50 mM KH2P04, 100 mM KCl, pH 7.0) and in aqueous buffer containing 50% TFE. The CD spectrum of peptide CVX0270 under benign conditions at 20° C. is shown in FIG. 7. The CD spectra is typical of a helical peptide with minima at 222 nm and 209 nm and high positive ellipticity below 200 nm. Typically, the molar ellipticity at 222 nm ([θ]₂₂₂) has been used to measure helical content in peptide. For peptide CVX0270 this corresponds to a value of −29800 which indicates that this peptide is predominantly α-helical. Theoretically, the [θ]₂₂₂ value for a peptide of 27 residues is −33900 (Chen et al., 1974). Therefore peptide CVX0270 is 88% helical. It should be noted that the linker region (Cys-Nle-Gly) is designed not to be helical and therefore decreases the [θ]₂₂₂ signal. The ratio of the molar ellipticity at 222 and 208 ([θ]₂₂₂/[θ]₂₀₈) is greater than 1.02 and similar to that observed before for coiled-coils (Hodges et al., 1988; Lau et al, 1984; Zhou et al., 1992) and distinctly different from non interacting α-helices in which the [θ]₂₀₈ is greater than the [θ]₂₂₂. In the presence of 50% TFE, helical content increased slightly to 101%. The data indicate that peptide CVX0270 is highly helical and found predominantly in the coiled-coil conformation under aqueous conditions.

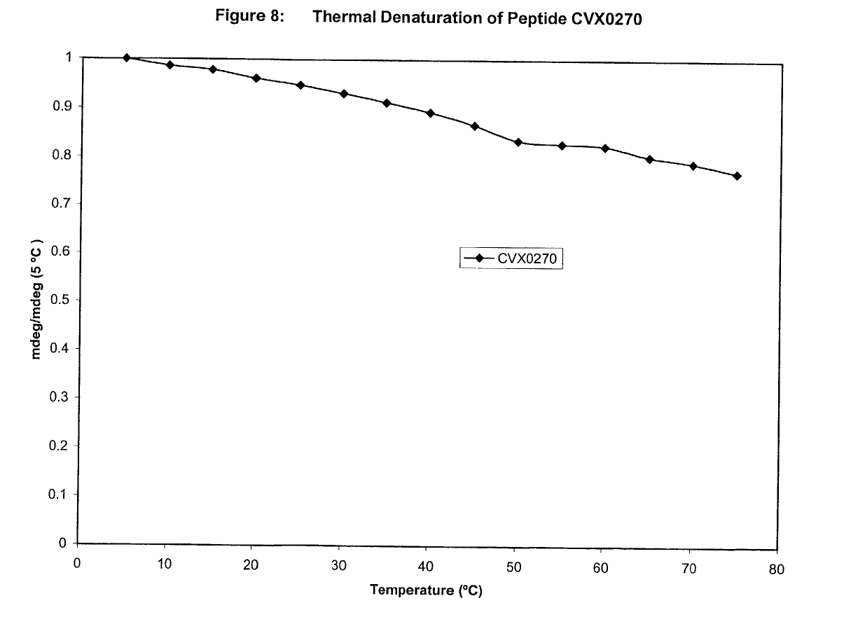

In order to determine the stability of CVX0270, a thermal denaturation study was undertaken. FIG. 8 shows the denaturation curve obtained by monitoring the [θ]₂₂₂ as a function of temperature. The study indicates that peptide CVX0270 is very stable with the peptide exhibiting 77% of its original helicity at 75° C. The above results demonstrate that the coiled-coil forming sequence with isoleucine at a positions and leucine residues at d positions is sufficiently stable to house a helical epitope from another protein sequence.

### Example 4

### Immunogenicity of Coiled-Coil Proteins

The peptide “Strep” (SEQ ID NO: 7) as described in Example 3 was coupled to the protein carrier tetanus toxoid (TT) as described in Materials and Methods. Immunogenicity of this peptide was determined as follows.

Balb/c mice (6-8 weeks, female, Charles River) were immunized on day 0 (1° primary immunization), day 7 (2° secondary immunization), day 28 (3° tertiary immunization), and day 42 (boost) by intraperitoneal injection (100 μL total, 50% Alhydrogel 2% adjuvant, Cedarlane, Catalog # SF2000-250) with Strep-tetanus toxoid (20, 10 or 5 μg) and with unconjugated tetanus toxoid. These antigens were diluted to various doses in 0.9% NaCl and mice injected with 0.9% NaCl were used as negative controls. Mice were bled following the 1°, 2°, 3° and boost injections on days 6, 14, 35 and 49 to collect serum to assay for antibody titers (direct ELISA) and antibody isotype response (isotyping ELISA), determine antibody specificity (inhibition ELISA) and measure immunoprotective antibody responses (bactericidal/opsonization assays).

A typical immunization schedule is shown in Table 1. Various other immunization schedules and adjuvant formulations known in the art would also be effective. Various delivery methods including intravascular, intramuscular, intradermal, subcutaneous, oral, nasal and aerosol inhalation routes would also be effective.

TABLE 1
BALB/c Mouse immunized I.P. - Strep-TT
Immunization Schedule

| Day | Procedure |
|---|---|
| 0 | Immunize i.p. |
| 6 | Bleed |
| 7 | Immunize i.p. |
| 14 | Bleed |
| 28 | Immunize i.p. |
| 35 | Bleed |
| 42 | Immunize i.p. |
| 49 | Cardiac Bleed |

### Study Groups

### (10 A.BY mice/group)

1. Strep−TT (20 μg/mouse)+adjuvant

2. Strep−TT (10 μg /mouse)+adjuvant

3. Strep−TT (5 μg/mouse)+adjuvant

4. Tetanus toxoid (20 μg/mouse)+adjuvant

5. PBS (without Alum)

### A. Determining Antibody Levels Elicited by Coiled-Coil Antigen Conjugates

The basic procedure to measure antibody levels is by the following direct ELISA protocol:

1. Coat EIA plates (COSTAR) with 1.0 μg/mL (0.1 μg per well) of antigen in 0.05 M carbonate-bicarbonate buffer (pH 9.6, 100 μL/well).

2. Incubate the plate(s) at 4° C. overnight.

3. On the next day, wash plates 3× with washing buffer (PBS/0.05% Tween). Flick off excess liquid by tapping the plates on the bench top.

4. Block plates with 100 μL blocking buffer per well (PBS/2% BSA). Incubate plates for 1 hour at 37° C.

5. Wash plates 5× with washing buffer (PBS/0.05% Tween). Flick off excess liquid by tapping the plates on the bench top.

6. Add 100 μL per well of test antibody appropriately diluted in dilution buffer (PBS/0.1% Tween). Incubate plates for 60 minutes at 37° C.

7. Wash plates 3× with washing buffer (PBS/0.05% Tween). Flick off excess liquid by tapping the plates on the bench top.

8. Dilute HRP anti-mouse IgG (Jackson Lab) in dilution buffer (PBS/0.1% Tween) to a concentration of 1:5000. Add 100 μL per well and incubate at 37° C. for 60 minutes.

9. Wash plates 3× with washing buffer (PBS/0.05% Tween). Flick off excess liquid by tapping the plates on the bench top.

10. Prepare HRP substrate; 0.03% Hydrogen peroxide (30%) and 1 mM ABTS dissolved in 0.01 M Sodium citrate buffer (Sodium citrate (1.48 g) dissolve sodium citrate in 500 mL distilled H₂O. Citric acid (1.58 g) dissolve citric acid in 750 mL distilled H₂O. Pour citric acid solution into sodium citrate solution to get a pH 4.2). Add 100 μL to each well and develop in a dark place for 30 minutes.

11. Read the absorbance with an ELISA plate reader at 405 nm at 30 minutes.

The mean O. D. ELISA readings obtained with antisera to the Strep-TT conjugates (20 μg, 10 μg and 5 μg) is shown in Table 2. The Strep-TT conjugate at all three dose concentrations (20 μg, 10 μg and 5 μg) elicited significant antibody titers to the Strep (EIEALKKKIEELSDKIDELEKEIK) hapten. These antibodies were immunogen specific, as the unconjugated tetanus toxoid did not elicit any antibodies or cross-reactive antibodies to the coiled-coil Strep antigen.

TABLE 2
ELISA Results of Mouse Serum Antibodies to the
Coiled-Coil Strep Antigen

| | Murine Sera (Day 49) (OD 405 nm, | | | | |
|---|---|---|---|---|---|
| | Strep-BSA coating antigen) | | | | |
| Serum | Strep-TT | Strep-TT | Strep-TT | | |
| Dilutions | (20 μg)* | (10 μg)* | (5 μg)* | TT* | PBS* |
| 1:2000 | 3.75 | 3.71 | 3.86 | 0.11 | 0.01 |
| 1:4000 | 3.57 | 3.60 | 3.61 | 0.04 | 0.00 |
| 1:8000 | 2.48 | 2.66 | 2.60 | 0.01 | 0.00 |
| 1:16000 | 1.14 | 1.59 | 1.50 | 0.06 | 0.00 |
| 1:32000 | 0.76 | 0.84 | 0.95 | 0.00 | 0.00 |
| 1:64000 | 0.36 | 0.42 | 0.41 | 0.00 | 0.00 |
| 1:128000 | 0.18 | 0.23 | 0.22 | 0.00 | 0.00 |
| 1:256000 | 0.04 | 0.10 | 0.12 | 0.00 | 0.00 |

*mice were i.p. injected on day 0, 7, 28 and 42 with Strep-TT + adjuvant, TT + adjuvant or PBS. Sera were collected on day 49.

### B. Determining Antibody Isotype Levels Elicited by Coiled-Coil Antigen Conjugates

The basic procedure to measure antibody isotype levels is as follows to quantify IgM, IgG, and IgA isotypes elicited by coil-coiled antigen conjugates.

1. Coat EIA plates (COSTAR) with 1.0 μg/mL (0.1 μg per well) of antigen in 0.05 M carbonate-bicarbonate buffer pH 9.6, 100 μL/well.

2. Incubate the plate(s) at 4° C. overnight.

3. On the next day, wash plates 3× with washing buffer (PBS/0.05% Tween). Flick off excess liquid by tapping the plates on the bench top.

4. Block plates with 100 μL blocking buffer per well (PBS/2% BSA). Incubate plates for 1 hour at 37° C.

5. Wash plates 5× with washing buffer (PBS/0.05% Tween). Flick off excess liquid by tapping the plates on the bench top.

6. Prepare 1:250 dilution mouse serum in the working buffer (PBS/0. 1% Tween). Add 100 μL/well into the appropriate well. Incubate plates for 1 hour at 37° C.

7. Wash plates 3× with washing buffer (PBS/0.05% Tween). Flick off excess liquid by tapping the plates on the bench top.

8. Dilute HRP-labeled detection antibodies (Southern Biotechnology Associates Inc.). add 100 μL/well into the appropriate well. Incubate plates for 1 hour at 37° C.

9. Wash plates 3× with washing buffer (PBS/0.05% Tween). Flick off excess liquid by tapping the plates on the bench top.

10. Prepare HRP substrate; 0.03% Hydrogen peroxide (30%) and 1mM ABTS dissolved in 0.01 M Sodium citrate buffer (Sodium citrate (1.48 g) dissolve sodium citrate in 500 mL distilled H₂O. Citric acid (1.58 g) dissolve citric acid in 750 mL distilled H₂O. Pour citric acid solution into sodium citrate solution to get a pH 4.2). Add 100 μL to each well and develop in a dark place for 30 minutes

11. Read the absorbance with an ELISA plate reader at 405 nm at 30 minutes.

The IgG antibody isotype response to the Strep-TT coiled-coil antigen is shown in Table 3. These results demonstrate the presence of significant IgG1 antibody levels to the Strep coiled-coil antigen after secondary immunization. A significant IgG isotype switch from IgG1 to IgG₂ₐ, IgG_{2b} and IgG₃ was seen after the booster injection (Day 49 bleed). The observed IgG antibody maturation to the coiled-coil Strep antigen is typical of a TD response.

TABLE 3
Isotyping ELISA of Mouse Serum Antibodies
to the Coiled-Coil Strep Antigen
DAY 6

Isotypes (O.D. 405 nm)

| Antiserum to:* | IgG₁ | IgG₂ₐ | IgG_{2b} | IgG₃ |
|---|---|---|---|---|
| Strep-TT (20 μg) | 0.14 | 0.01 | 0.04 | 0.04 |
| Strep-TT (10 μg) | 0.12 | 0.01 | 0.03 | 0.04 |
| Strep-TT (5 μg) | 0.14 | 0.05 | 0.08 | 0.10 |
| TT (20 μg) | 0.06 | 0.02 | 0.01 | 0.01 |
| PBS | 0.03 | 0.00 | 0.00 | 0.01 |

*mice were i.p. injected on day 0 with Strep-TT + adjuvant, TT +
adjuvant or PBS. Sera were collected on day 6.

DAY 14

Isotypes (O.D. 405 nm)

| Antiserum to:* | IgG₁ | IgG₂ₐ | IgG_{2b} | IgG₃ |
|---|---|---|---|---|
| Strep-TT (20 μg) | 2.00 | 0.71 | 0.95 | 1.08 |
| Strep-TT (10 μg) | 1.84 | 0.91 | 1.01 | 2.22 |
| Strep-TT (5 μg) | 1.69 | 0.62 | 0.95 | 1.23 |
| TT (20 μg) | 0.05 | 0.00 | 0.00 | 0.01 |
| PBS | 0.03 | 0.00 | 0.00 | 0.00 |

*mice were i.p. injected on day 0 and 7 with Strep-TT + adjuvant,
TT + adjuvant or PBS. Sera were collected on day 14

Day 35

Isotypes (O.D. 405 nm)

| Antiserum to:* | IgG₁ | IgG₂ₐ | IgG_{2b} | IgG₃ |
|---|---|---|---|---|
| Strep-TT (20 μg) | 2.24 | 0.40 | 0.49 | 0.45 |
| Strep-TT (10 μg) | 2.07 | 0.56 | 0.59 | 1.00 |
| Strep-TT (5 μg) | 2.16 | 0.60 | 0.76 | 0.75 |
| TT (20 μg) | 0.08 | 0.01 | 0.01 | 0.01 |
| PBS | 0.01 | 0.00 | 0.00 | 0.01 |

*mice were i.p. injected on day 0, 7 and 28 with Strep-TT + adjuvant,
TT + adjuvant or PBS. Sera were collected on day 35.

Day 49

Isotypes (O.D. 405 nm)

| Antiserum to:* | IgG₁ | IgG₂ₐ | IgG_{2b} | IgG₃ |
|---|---|---|---|---|
| Strep-TT (20 μg) | 3.91 | 1.14 | 1.28 | 0.74 |
| Strep-TT (10 μg) | 3.85 | 1.35 | 2.01 | 1.28 |
| Strep-TT (5 μg) | 3.88 | 2.10 | 2.96 | 0.82 |
| TT (20 μg) | 0.44 | 0.03 | 0.02 | 0.05 |
| PBS | 0.01 | 0.01 | 0.01 | 0.02 |

*mice were i.p. injected on day 0, 7, 28 and 42 with Strep-TT + adjuvant, TT + adjuvant or PBS. Sera were collected on day 49.

### C. Determining Antibody Binding in Competition with Coiled-Coil Antigen Conjugates or Free Peptides

The specificity of the antisera was further examined by inhibition ELISA using peptides with or without a coiled-coil structure. The protocol for this inhibition ELISA is as follows:

1. Dilute the coating antigen to 1.0 μ/mL (0.1 μg per well) in carbonate-bicarbonate buffer. Use glass tubes.

2 Add 100 μL of the coating antigen to each well of the plate. Store the plate(s) overnight at 4° C.

3. Shake out the wells and tap upside-down onto Kimwipes. Wash plate three times with ˜200 μL wash buffer (PBS/0.05% Tween)(v/v) per well.

4 Add 200 μL blocking buffer per well (PBS/2% BSA)(w/v). Incubate plates for 60 minutes at 37° C.

5. Shake out the wells and tap upside-down onto Kimwipes. Wash plate five times with5 ˜200 μL wash buffer (PBS/0.05% Tween)(v/v) per well.

6. Add 50 μL of selected competitive conjugates or free peptides appropriately diluted in dilution buffer (PBS/0.1% Tween)(v/v). (NOTE: the competitive peptide has a ×2 concentration in 50 AL).

7. Added 50 μL per well of test antibody appropriately diluted in dilution buffer (PBS/0.1% Tween)(v/v). (NOTE: the test antibody has a ×2 concentration in 50 μL). The final volume in each well was 100 μL.

8. Incubate plates for 60 minutes at 37° C. with slow shaking on a ELISA shaker.

9 . Shake out the wells and tap upside-down onto Kimwipes. Wash plate three times with ˜200 μL wash buffer (PBS/0.05% Tween)(v/v) per well. Dilute Peroxidase-conjugated anti-mouse IgG in dilution buffer (PBS/0.1% Tween)(v/v) to a concentration of 1:5000. Add 100 μL per well and incubate at 37° C. for 60 minutes.

10. Shake out the wells and tap upside-down onto Kimwipes. Wash plate three times with ˜200 μL wash buffer (PBS/0.05% Tween)(v/v) per well.

11. Prepare HRP substrate; 0.03% Hydrogen peroxide (30%) and 1 mM ABTS dissolved in 0.01 M Sodium citrate buffer (Sodium citrate (1.48 g) dissolve sodium citrate in 500 mL distilled H₂O. Citric acid (1.58 g) dissolve citric acid in 750 mL distilled H₂O. Pour citric acid solution into sodium citrate solution to get a pH 4.2). Add 100 μL to each well and develop in a dark place for 30 minutes

12. Read the absorbance with an ELISA plate reader at 405 nm at 30 minutes.

The sequences of the Strep #1, #2 and #3 free peptides used in this inhibition assay are:

Strep #1: Coiled-coil Strep antigen with lactams EIEALKKKIEELSDKIDELEKEIK

Strep #2: Single stranded Strep antigen with lactams EEESDKKDEEDAEKAE

Strep #3: Coiled coil Strep antigen EIEALKKKIEELSDKIDELEKEIK

The residues that make up the lactam bridges are highlighted in a bold font. The lactams are formed by reacting the carboxylic acid side chains of glutamic acids residues with the side chain amino groups of lysine residues. These lactams stabilize α-helices and coiled coils when they are situated 4 residues apart and when they are oriented in the glutamic acid to lysine direction.

The epitope is highlighted in italicized script. From all three sequences it can be seen that the epitope begins with two glutamic acid residues (EE) followed by a leucine residue (L). In the case of the single stranded peptide the leucine residue is replaced by a glutamic acid (E) residue which is part of the lactam bridge. The epitope continues with SKD followed by an isoleucine (I) in the coiled-coils and the lysine residue (K) which is involved in the lactam bridge in the single stranded peptide. The final two residues of the epitope are DE.

Table 4 shows the inhibition ELISA results of anti-Strep serum using 3 different Strep peptides, the Strep-TT immunogen, TT and BSA. The Strep #3 peptide (peptide used as the inmunogen) showed significant inhibition, the Strep #2 peptide (linear peptide, which has low helical content) showed no inhibition. Therefore, it would seem that the immunogen Strep-TT elicited antibodies which recognize conformational (or 3D) epitope structures. The Strep-TT immunogen did not elicit antibodies to the linear epitope. These results therefore suggest that coiled-coil antigens elicit antibodies which recognize conformational epitopes. Inhibition was also seen with Strep #1 peptide, which peptide contains lactam bridges which adds further stability to the coiled-coil antigen.

TABLE 4
Inhibition ELISA of Anti-Strep.(1:25,000 dilution, Day
49) Using Various Strep Antigen Inhibitors

| Conc. | O.D. 405 nm | | | | | |
|---|---|---|---|---|---|---|
| (μg per | Strep. | Strep. | Strep. | Strep.- | | |
| 100 μL) | #1 | #2 | #3 | T.T. | T.T. | BSA |

Anti-Strep.-T.T. (20 μg Dose)

| | | | | | | |
|---|---|---|---|---|---|---|
| 10 | 0.663 | 1.266 | 0.110 | 0.028 | 1.342 | 1.399 |
| 5 | 0.720 | 1.186 | 0.143 | 0.026 | 1.051 | 1.485 |
| 2.5 | 0.823 | 1.173 | 0.254 | 0.056 | 1.412 | 1.435 |
| 1 | 0.959 | 1.164 | 0.555 | 0.094 | 1.457 | 1.442 |
| 0.5 | 1.081 | 1.144 | 0.776 | 0.109 | 1.564 | 1.187 |
| 0.25 | 1.237 | 1.251 | 1.036 | 0.165 | 1.638 | 1.510 |

Anti-Strep.-T.T. (10 μg Dose)

| | | | | | | |
|---|---|---|---|---|---|---|
| 10 | 0.941 | 1.210 | 0.055 | 0.010 | 1.820 | 1.784 |
| 5 | 0.917 | 1.258 | 0.233 | 0.010 | 1.521 | 1.790 |
| 2.5 | 0.963 | 1.232 | 0.269 | 0.015 | 1.726 | 1.765 |
| 1 | 1.129 | 1.312 | 0.619 | 0.065 | 1.802 | 1.670 |
| 0.5 | 1.256 | 1.293 | 0.872 | 0.068 | 1.800 | 1.422 |
| 0.25 | 1.340 | 1.279 | 1.123 | 0.113 | 1.871 | 1.845 |

Anti-Strep.-T.T. (5 μg Dose)

| | | | | | | |
|---|---|---|---|---|---|---|
| 10 | 0.872 | 1.294 | 0.009 | 0.000 | 1.803 | 1.740 |
| 5 | 0.960 | 1.318 | 0.090 | 0.000 | 1.109 | 1.571 |
| 2.5 | 1.087 | 1.233 | 0.213 | 0.000 | 1.822 | 1.761 |
| 1 | 1.125 | 1.322 | 0.501 | 0.053 | 1.797 | 1.572 |
| 0.5 | 1.289 | 1.377 | 0.746 | 0.064 | 1.836 | 1.717 |
| 0.25 | 1.388 | 1.303 | 1.001 | 0.107 | 1.925 | 1.786 |

Strep. #1 = Coiled-coil + lactam bridges
Strep. #2 = Linear sequence
Strep. #3 = SEQ ID NO:7

### D. Bactericidal and Opsonization Assays to Measure Immunoprotective Antibodies Elicited by Coil-Coiled Antigen Conjugates

The basic bactericidal and opsonization assays used are as follows:

Bactericidal Assay

1. Streak an agar plate with desired gram positive bacteria procured from various culture collections (ATCC). Incubate at 37° C. overnight.

2. On the next day, pick an isolated colony and inoculate it in 1.0 ml of Todd-Hewitt Broth (THB)+Yeast Extraction (YE) media in a sterile test tube. Incubate at 37° C. overnight.

3. On the following day, measure O. D. of inoculated bacteria at 420 nm wavelength. Use THB+YE media as blank.

4. To a sterile flat bottom 96-well plate, add a sterile 2.5 mm glass bead in each well.

5. To each well, add:

5 μL of bacteria

10 μL of mouse serum to be tested incubate at 37° C. for 1 hour.

NOTE: Step 5 and 6 are done in triplicate.

6. After 1 hour incubation, prepare 1:20 dilution exogenous complement (Low-Tox guinea pig Complement, Cedarlane) sterilely in THB+YE. Add 50 μL/well. Incubate at 37° C. for 1 hour.

7. After complement incubation, 50 μL aliquot is plated out on agar plates using glass spreader.

8. Wrap all agar plates in plastic bags and incubate at 37° C. overnight.

9. On the next day, count colony forming units (CFU).

Opsonization Assay

1. Streak an agar plate with desired gram positive bacteria procured from various culture collections (ATCC). Incubate at 37° C. overnight.

2. On the next day, pick an isolated colony and inoculate it in 1.0 ml of Todd-Hewitt Broth (THB)+Yeast Extraction (YE) media in a sterile test tube. Incubate at 37° C. overnight.

3. The next day prepare 100 U/ml of sterile heparin.

4. I.V. inject 100 μL of sterile heparin into tail vein of each mouse (5-10). After 10 minutes, cardiac bleed mice into a sterile tube.

5. Measure O. D. of inoculated bacteria at 420 nm wavelength. Use THB+YE media as blank.

6. To a sterile flat bottom 96-well plate, add a sterile 2.5 mm glass bead in each well.

7. To each well, add:

50 μL of heparinized blood

10 μL of mouse serum to be tested

5 μL of bacteria

incubate at 37° C. for 1 hour.

NOTE: Do this step in triplicate.

8. After 1 hour incubation, prepare 1:20 dilution exogenous complement (Low-Tox guinea pig Complement, Cedarlane) sterilely in distilled water. Add 50 μL/well. Incubate at 37° C. for 1 hour on a shaker (slow motion).

9. After one hour, 100 μL aliquot is plated out on agar plates using glass spreader.

10. Wrap all agar plates in plastic bags and incubate at 37° C. overnight.

11. On the next day, count colony forming units (CFU).

Sera from mice immunized with the Strep-TT conjugates (20, 10, 5 μg doses) were found to be immunoprotective as measured by the opsonization assay. Antisera to Strep-TT showed reduction in colony forming units of representative Streptococcus pneumoniae serotypes (1, 3, and 5). Little or no reduction in colony forming units was seen with sera from mice injected with tetanus toxoid or with PBS. The Strep-TT conjugate elicited immunoprotective antibodies to coiled-coil antigens of S. pneumoniae.

TABLE 5
Opsonization Results of Antiserum to Strep-TT (Day 49) using
Streptococcus pneumoniae serotypes (1, 3 and 5)

| | Percent Reduction of CFU | | |
|---|---|---|---|
| Antiserum to: | Type 1 | Type 3 | Type 5 |
| Strep-TT (20 μg) | 58 | 49 | 45 |
| Strep-TT (10 μg) | 22 | 50 | 24 |
| Strep-TT (5 μg) | 30 | 48 | 24 |
| TT | 7 | 6 | 7 |
| PBS | 0 | 0 | 1 |

[image]

[image]

[image]

[image]

[image]

[image]

[image]

We claim:

1. A synthetic peptide of the formula I:
(AXXDXXX)ₙ I
Wherein
A is Ile, Leu, Val or a derivative thereof;
D is Leu, Ile, Val or a derivative thereof;
each X is an amino acid residue or derivative thereof which corresponds to an amino acid residue of an epitope of a native coiled-coil protein;
the X residues in each (AXXDXXX) repeat form a set of X residues; and n is equal to or greater than 1.

2. The peptide of claim 1 wherein A is Ile and D is Leu in every (AXXDXXX) repeat.

3. The peptide of claim 1 wherein n is about 3 to 6.

4. The peptide of claim 1 wherein said X residues are amino acids that are solvent exposed in an coiled-coil region of the native protein.

5. The peptide of claim 1 wherein each of said sets of X residues is from the same epitope of a single protein.

6. The peptide of claim 1 which contains at least two different sets of X residues.

7. The peptide of claim 6 wherein each of said different sets is independently selected from the group consisting of different epitopes of the same protein and epitopes from different proteins.

8. The peptide of claim 1 which further comprises additional amino acids at the C-terminus and/or N-terminus of the peptide.

9. The peptide of claim 8 wherein said additional amino acid residues are CNleG at the N-terminus of the peptide.

10. The peptide of claim 1 wherein the set of X residues correspond to a consensus sequence of solvent exposed residues of native coiled-coil proteins.

11. The peptide of claim 10 wherein the coiled-coil proteins are selected from the group consisting of Pneumococcal surface protein A, Pneumococcal surface protein C, and Pneumococcal adhesin A.

12. The peptide of claim 11 wherein the peptide comprises an amino acid sequence selected from the group consisting of
EELX₁X₂KIDELDX₃EIAX₄LEKX₅ (SEQ ID NO: 5) and
EELX₁X₂KIDELD (1-11 of SEQ ID NO: 5), wherein X₁, X₂, X₃, X₄ or X₅ is any amino acid.

13. The peptide of claim 12 wherein
X₁ is S, Q, N or D;
X₂ is D, N or K;
X₃ is A or N;
X₄ is K, E or D; and
X₅ is N, D or E.

14. A synthetic peptide of the formula I:
(AXXDXXX)ₙ I
Wherein
A is Ile, Leu, Val or a derivative thereof;
D is Leu, Ile, Val or a derivative thereof;
each X is an amino acid residue or derivative thereof which corresponds to an amino acid residue of an epitope of a native coiled-coil protein, except at least one X is replaced with a charged amino acid residue in a manner which allows a salt bridge to form between the charged amino acid and another amino acid residue of an opposite charge, which salt bridge facilitates the peptide to assume a coiled-coil structure;
the X residues in each (AXXDXXX) repeat form a set of X residues; and
n is equal to or greater than 1.

15. A peptide of claim 14 wherein the charged amino acid is selected from the group consisting of Asp, Glu, Lys, Arg and His.

16. A method of making a peptide of the formula I comprising:
a) selecting an epitope of a coiled-coil protein;
b) determining which amino acid residues of said epitope are solvent exposed; and
c) inserting said solvent exposed amino acid residues into the X positions of formula I.

17. The method of claim 16 wherein the coiled-coil protein is a microbial protein.

18. The method of claim 16 wherein the selection of epitopes is performed using a computer algorithm.

19. The method of claim 16 wherein more than one set of epitopic amino acids is used.

20. The method of claim 19 wherein each of said sets is independently selected from the group consisting of different epitopes of the same protein and epitopes from different proteins.

21. A composition useful to stimulate an immune response in an animal, said composition comprising at least one peptide of formula I.

22. The composition of claim 21 wherein the peptide of formula I is conjugated to a carrier protein.

23. The composition of claim 21 further comprising an adjuvant.

24. The composition of claim 21 which contains at least two different sets of X residues.

25. The composition of claim 24 wherein each of said different sets is independently selected from the group consisting of different epitopes of the same protein and epitopes from different proteins.

26. The composition of claim 24 which is useful to stimulate an immune response to more than one strain and/or species of microorganism.

27. A method of eliciting an immune response in an animal, comprising administering a peptide of the formula I to said animal.

28. An antibody which recognizes a peptide of the formula I.

29. The antibody of claim 28 wherein the peptide of the formula I contains solvent exposed amino acids from a microbial protein.

30. The antibody of claim 28 which binds to more than one strain and/or species of microorganism.

31. The antibody of claim 28 which is polyclonal or monoclonal.

32. A pharmaceutical composition comprising an antibody according to claim 28.

33. The composition of claim 32 which further comprises a pharmaceutically acceptable excipient or carrier.

34. An antibody produced by administering a peptide of the formula I to an animal so as to stimulate an immune response.

35. A composition useful as a vaccine, wherein said composition comprises a peptide of formula I.

36. The composition of claim 35 wherein more than one set of epitopic amino acids is used in the peptide of formula I.

37. The composition of claim 36 wherein the sets of epitopic amino acids are from different strains and/or species of microorganism.

38. The composition of claim 35 which provides cross protection to more than one strain and/or species of microorganism.

39. The composition of claim 36 which provides cross protection to more than one strain and/or species of microorganism.

40. The composition of claim 37 which provides cross protection to more than one strain and/or species of microorganism.

41. The composition of claim 35 which further comprises a pharmaceutically acceptable excipient or carrier.

42. A method of preventing a microbial infection comprising administering to a mammal susceptible to said infection a peptide of formula I.

43. The method of claim 42 wherein more than one set of epitopic amino acids is used in the peptide of formula I and the sets of epitopic amino acids are from different strains and/or species of microorganism.

44. The method of claim 42 which is useful to prevent infection by several strains and/or species of microorganism.

45. The method of claim 43 which is useful to prevent infection by several strains and/or species of microorganism.

46. A method of treating or preventing microbial infection in an animal susceptible to or suffering from such infection, comprising administering to said animal an effective amount of an antibody to a microbial protein, wherein said antibody is produced by administering a peptide of formula I to an animal.

47. The method of claim 46 which prevents symptoms of infection in said animal.

48. The method of claim 46 which is useful to treat or prevent infection by several strains and/or species of microorganism.

49. A method of determining the presence of a particular microorganism in a sample comprising:
a) contacting the sample with an antibody to a peptide of formula I which peptide comprises epitopes from the particular microorganism; and
b) determining whether said antibody binds to a component of said sample.

50. The method of claim 49 wherein the sample is a biological sample.

51. The method of claim 49 which is used to determine the causative agent of a microbial infection.

52. The method of claim 51 which is used to simultaneously detect the presence of several strains and/or species of microorganism in tile sample.

53. The method of claim 50 which is used to simultaneously detect the presence of several strains and/or species of microorganism in the sample.

54. The method of claim 51 which is used to simultaneously detect the presence of several strains and/or species of microorganism in the sample.

55. A method for determining the presence of antibodies to a microbial protein in a biological sample, comprising:
a) contacting said biological sample with a peptide of formula I, which peptide comprises at least one epitope from said microbial protein; and
b) determining whether antibodies in said biological sample bind to said peptide.

56. The method of claim 55 which is used to determine prior exposure of an animal to a particular microorganism.

57. The protein of claim 8 wherein the additional amino acids stabilize the peptide through the formation of lactam bridges.
